(19) United States
(12) Patent Application Publication
(10) Pub. No.: US 2003/0084473 A1
(43) Pub. Date: May 1, 2003

(54) Non-transgenic herbicide resistant plants

(75) Inventors: Greg Gocal, San Diego, CA (US); Patricia Avissar, Durham, NC (US); Mark Knuth, Poway, CA (US); Peter Beetham, Carlsbad, CA (US); Keith Walker, San Diego, CA (US)

Correspondence Address: SUGHRUE MION, PLLC, 1010 EL CAMINO REAL, SUITE 300, MENLO PARK, CA, 94025, US

(73) Assignee: VALIGEN

(21) Appl. No.: 10/214,766

(22) Filed: Aug. 9, 2002

Related U.S. Application Data
(60) Provisional application No. 60/311,734, filed on Aug. 9, 2001.

(51) Int. Cl. A01H 1/00; C12N 9/10; C12N 15/82; C12N 5/04

(52) U.S. Cl. 800/278; 435/193; 435/419

(57) ABSTRACT

The present invention relates to the production of a non-transgenic plant resistant or tolerant to a herbicide of the phosphonomethylglycine family, e.g., glyphosate. The present invention also relates to the use of a recombinagenic oligonucleobase to make a desired mutation in the chromosomal or episomal sequences of a plant in the gene encoding for 5-enol pyruvylshikimate-3-phosphate synthase (EPSPS). The mutated protein, which substantially maintains the catalytic activity of the wild-type protein, allows for increased resistance or tolerance of the plant to a herbicide of the phosphonomethylglycine family, and allows for the substantially normal growth or development of the plant, its organs, tissues or cells as compared to the wild-type plant irrespective of the presence or absence of the herbicide. The present invention also relates to a non-transgenic plant cell in which the EPSPS gene has been mutated, a non-transgenic plant regenerated therefrom, as well as a plant resulting from a cross using a regenerated non-transgenic plant having a mutated EPSPS gene. The amino acids at the positions 126, 177, 207, 438, 479, 480 and/or 505 are changed tp produce a mutant EPSPS gene product.

### 1. FIELD OF THE INVENTION

The present invention relates to the production of a non-transgenic plant resistant or tolerant to a herbicide of the phosphonomethylglycine family, e.g., glyphosate. The present invention also relates to the use of a recombinagenic oligonucleobase to make a desired mutation in the chromosomal or episomal sequences of a plant in the gene encoding for 5-enol pyruvylshikimate-3-phosphate synthase (EPSPS). The mutated protein, which substantially maintains the catalytic activity of the wild-type protein, allows for increased resistance or tolerance of the plant to a herbicide of the phosphonomethylglycine family, and allows for the substantially normal growth or development of the plant, its organs, tissues or cells as compared to the wild-type plant irrespective of the presence or absence of the herbicide. The present invention also relates to a non-transgenic plant cell in which the EPSPS gene has been mutated, a non-transgenic plant regenerated therefrom, as well as a plant resulting from a cross using a regenerated non-transgenic plant having a mutated EPSPS gene.

### 2. BACKGROUND TO THE INVENTION

2.1 Phosphonomethylglycine Herbicides

Herbicide-tolerant plants may reduce the need for tillage to control weeds thereby effectively reducing soil erosion. One herbicide which is the subject of much investigation in this regard is N-phosphonomethylglycine, commonly referred to as glyphosate. Glyphosate inhibits the shikimic acid pathway which leads to the biosynthesis of aromatic compounds including amino acids, hormones and vitamins. Specifically, glyphosate curbs the conversion of phosphoenolpyruvic acid (PEP) and 3-phosphoshikimic acid to 5-enolpyruvyl-3-phosphoshikimic acid by inhibiting the enzyme 5-enolpyruvylshikimate-3-phosphate synthase (hereinafter referred to as EPSP synthase or EPSPS). For purposes of the present invention, the term “glyphosate” includes any herbicidally effective form of N-phosphonomethylglycine (including any salt thereof), other forms which result in the production of the glyphosate anion in plants and any other herbicides of the phosphonomethlyglycine family.

Tolerance of plants to glyphosate can be increased by introducing a mutant EPSPS gene having an alteration in the EPSPS amino acid coding sequence into the genome of the plant. Examples of some of the mutations in the EPSPS gene for inducing glyphosate tolerance are described in the following patents: U.S. Pat. No. 5,310,667; U.S. Pat. No. 5,866,775; U.S. Pat. No. 5,312,910; U.S. Pat. No. 5,145,783. These proposed mutations typically have a higher Kᵢ for glyphosate than the wild-type EPSPS enzyme which confers the glyphosate-tolerant phenotype, but these variants are also characterized by a high Kₘ for PEP which makes the enzyme kinetically less efficient (Kishore et al., 1988, Ann. Rev. Biochem. 57:627-663; Schulz et al., 1984, Arch. Microbiol. 137: 121-123; Sost et al., 1984, FEBS Lett. 173: 238-241; Kishore et al., 1986, Fed. Proc. 45: 1506; Sost and Amrhein, 1990, Arch. Biochem. Biophys. 282: 433-436). Many mutations of the EPSPS gene are chosen so as to produce an EPSPS enzyme that is resistant to herbicides, but unfortunately, the EPSPS enzyme produced by the mutated EPSPS gene has a significantly lower enzymatic activity than the wild-type EPSPS. For example, the apparent Km for PEP and the apparent K₁ for glyphosate for the wild-type EPSPS from E. coli are 10 μM and 0.5 μM, while for a glyphosate-tolerant isolate having a single amino acid substitution of alanine for glycine at position 96, these values are 220 μM and 4.0 mM, respectively. A number of glyphosate-tolerant EPSPS genes have been constructed by mutagenesis. Again, the glyphosate-tolerant EPSPS had lower catalytic efficiency (Vₘₐₓ/Kₘ), as shown by an increase in the Kₘ for PEP, and a slight reduction of the Vₘₐₓ of the wild-type plant enzyme (Kishore et al., 1988, Ann. Rev. Biochem. 57:627-663).

Since the kinetic constants of the variant enzymes are impaired with respect to PEP, it has been proposed that high levels of overproduction of the variant enzyme, 40-80 fold, would be required to maintain normal catalytic activity in plants in the presence of glyphosate (Kishore et al., 1988, Ann. Rev. Biochem. 57:627-663). It has been shown that glyphosate-tolerant plants can be produced by inserting into the genome of the plant the capacity to produce a higher level of EPSP synthase in the chloroplast of the cell (Shah et al., 1986, Science 233, 478-481), which enzyme is preferably glyphosate-tolerant (Kishore et al., 1988, Ann. Rev. Biochem. 57:627-663).

The introduction of the exogenous mutant EPSPS genes into plant cells is well documented. For example, according to U.S. Pat. No. 4,545,060, to increase a plant's resistance to glyphosate, a gene coding for an EPSPS variant having at least one mutation that renders the enzyme more resistant to its competitive inhibitor, i.e., glyphosate, is introduced into the plant genome. However, many complications and problems are associated with these examples. Many such mutations result in low expression of the mutated EPSPS gene product or result in an EPSPS gene product with significantly lower enzymatic activity as compared to the wild type. The low expression and/or low enzymatic activity of the mutated enzyme results in abnormally low levels of growth and development of the plant.

While such variants in the EPSP synthases have proved useful in obtaining transgenic plants tolerant to glyphosate, it would be increasingly beneficial to obtain a variant EPSPS gene product that is highly glyphosate-tolerant but still kinetically efficient, such that improved tolerance can be obtained with a wild-type expression level.

2.2 Recombinagenic Oligonucleobases

Recombinagenic oligonucleobases and their use to effect genetic changes in eukaryotic cells are described in U.S. Pat. No. 5,565,350 to Kmiec (Kmiec I). Kmiec I teaches a method for introducing specific genetic alterations into a target gene. Kmiec I discloses, inter alia, recombinagenic oligonucleobases having two strands, in which a first strand contains two segments of at least 8 RNA-like nucleotides that are separated by a third segment of from 4 to about 50 DNA-like nucleotides, termed an “interposed DNA segment.” The nucleotides of the first strand are base paired to DNA-like nucleotides of a second strand. The first and second strands are additionally linked by a segment of single stranded nucleotides so that the first and second strands are parts of a single oligonucleotide chain. Kmiec I further teaches a method for introducing specific genetic alterations into a target gene. According to Kmiec I, the sequences of the RNA segments are selected to be homologous, i.e., identical, to the sequence of a first and a second fragment of the target gene. The sequence of the interposed DNA segment is homologous with the sequence of the target gene between the first and second fragment except for a region of difference, termed the “heterologous region.” The heterologous region can effect an insertion or deletion, or can contain one or more bases that are mismatched with the sequence of target gene so as to effect a substitution. According to Kmiec I, the sequence of the target gene is altered as directed by the heterologous region, such that the target gene becomes homologous with the sequence of the recombinagenic oligonucleobase. Kmiec I specifically teaches that ribose and 2′-O-methylribose, i.e., 2′-methoxyribose, containing nucleotides can be used in recombinagenic oligonucleobases and that naturally-occurring deoxyribose-containing nucleotides can be used as DNA-like nucleotides.

U.S. Pat. No. 5,731,181 to Kmiec (Kmiec II) specifically disclose the use of recombinagenic oligonucleobases to effect genetic changes in plant cells and discloses further examples of analogs and derivatives of RNA-like and DNA-like nucleotides that can be used to effect genetic changes in specific target genes. Other patents discussing the use of recombinagenic oligonucleobases include: U.S. Pat. Nos. 5,756,325; 5,871,984; 5,760,012; 5,888,983; 5,795,972; 5,780,296; 5,945,339; 6,004,804; and 6,010,907 and in International Patent No. PCT/US00/23457; and in International Patent Publication Nos. WO 98/49350; WO 99/07865; WO 99/58723; WO 99/58702; and WO 99/40789. Recombinagenic oligonucleobases include mixed duplex oligonucleotides, non-nucleotide containing molecules taught in Kmiec II and other molecules taught in the above-noted patents and patent publications.

Citation or identification of any reference in Section 2, or any section of this application shall not be construed as an admission that such reference is available as prior art to the present invention.

### 3. SUMMARY OF THE INVENTION

The present invention is directed to a non-transgenic plant or plant cell having one or more mutations in the EPSPS gene, which plant has increased resistance or tolerance to a member of the phosphonomethylglycine family and which plant exhibits substantially normal growth or development of the plant, its organs, tissues or cells, as compared to the corresponding wild-type plant or cell. The mutated gene produces a gene product having a substitution at one or more of the amino acid positions 126,177, 207, 438, 479,480 and 505 of the Arabidopsis EPSPS gene product or at an analogous amino acid position in an EPSPS homolog. The present invention is also directed to a non-transgenic plant having a mutation in the EPSPS gene, which plant is resistant to or has an increased tolerance to a member of the phosphonomethylglycine family, e.g., glyphosate, wherein the mutated EPSPS protein has substantially the same catalytic activity as compared to the wild-type EPSPS protein.

The present invention is also directed to a method for producing a non-transgenic plant having a mutated EPSPS gene that substantially maintains the catalytic activity of the wild-type protein irrespective of the presence or absence of a herbicide of the phosphonomethylglycine family. The method comprises introducing into a plant cell a recombinagenic oligonucleobase with a targeted mutation in the EPSPS gene and identifying a cell, seed, or plant having a mutated EPSPS gene.

Illustrative examples of a recombinagenic oligonucleobase are found in following patent publications, which are incorporated herein in their entirety by reference: U.S. Pat. Nos. 5,565,350; 5,756,325; 5,871,984; 5,760,012; 5,731,181; 5,888,983; 5,795,972; 5,780,296; 5,945,339; 6,004,804; and 6,010,907 and in International Patent No. PCT/US00/23457; and in International Patent Publication Nos. WO 98/49350; WO 99/07865; WO 99/58723; WO 99/58702; and WO 99/40789.

The plant can be of any species of dicotyledonous, monocotyledonous or gymnospermous plant, including any woody plant species that grows as a tree or shrub, any herbaceous species, or any species that produces edible fruits, seeds or vegetables, or any species that produces colorful or aromatic flowers. For example, the plant may be selected from a species of plant from the group consisting of canola, sunflower, tobacco, sugar beet, sweet potato, yam, cotton, maize, wheat, barley, rice, sorghum, tomato, mango, peach, apple, pear, strawberry, banana, melon, potato, carrot, lettuce, onion, soya spp, sugar cane, pea, peanut, field beans, poplar, grape, citrus, alfalfa, rye, oats, turf and forage grasses, flax, oilseed rape, cucumber, morning glory, balsam, pepper, eggplant, marigold, lotus, cabbage, daisy, carnation, tulip, iris, lily, and nut producing plants insofar as they are not already specifically mentioned.

The recombinagenic oligonucleobase can be introduced into a plant cell using any method commonly used in the art, including but not limited to, microcarriers (biolistic delivery), microfibers, electroporation, direct DNA uptake and microinjection.

The invention is also directed to the culture of cells mutated according to the methods of the present invention in order to obtain a plant that produces seeds, henceforth a “fertile plant”, and the production of seeds and additional plants from such a fertile plant including descendant (progeny) plants that contain the mutated EPSPS gene.

The invention is further directed to a method of selectively controlling weeds in a field, the field comprising plants with the disclosed EPSPS gene alterations and weeds, the method comprising application to the field of a herbicide to which the said plants have been rendered resistant.

The invention is also directed to novel mutations in the EPSPS gene and resulting novel gene product that confer resistance or tolerance to a member of the phosphonomethylglycine family, e.g., glyphosate, to a plant or wherein the mutated EPSPS has substantially the same enzymatic activity as compared to wild-type EPSPS.

3.1 Definitions

The invention is to be understood in accordance with the following definitions.

An oligonucleobase is a polymer of nucleobases, which polymer can hybridize by Watson-Crick base pairing to a DNA having the complementary sequence.

Nucleobases comprise a base, which is a purine, pyrimidine, or a derivative or analog thereof. Nucleobases include peptide nucleobases, the subunits of peptide nucleic acids, and morpholine nucleobases as well as nucleosides and nucleotides. Nucleosides are nucleobases that contain a pentosefuranosyl moiety, e.g., an optionally substituted riboside or 2′-deoxyriboside. Nucleosides can be linked by one of several linkage moieties, which may or may not contain a phosphorus. Nucleosides that are linked by unsubstituted phosphodiester linkages are termed nucleotides.

An oligonucleobase chain has a single 5′ and 3′ terminus, which are the ultimate nucleobases of the polymer. A particular oligonucleobase chain can contain nucleobases of all types. An oligonucleobase compound is a compound comprising one or more oligonucleobase chains that are complementary and hybridized by Watson-Crick base pairing. Nucleobases are either deoxyribo-type or ribo-type. Ribo-type nucleobases are pentosefuranosyl containing nucleobases wherein the 2′ carbon is a methylene substituted with a hydroxyl, alkyloxy or halogen. Deoxyribo-type nucleobases are nucleobases other than ribo-type nucleobases and include all nucleobases that do not contain a pentosefuranosyl moiety.

An oligonucleobase strand generically includes both oligonucleobase chains and segments or regions of oligonucleobase chains. An oligonucleobase strand has a 3′ end and a 5′ end. When a oligonucleobase strand is coextensive with a chain, the 3′ and 5′ ends of the strand are also 3′ and 5′ termini of the chain.

According to the present invention, substantially normal growth of a plant, plant organ, plant tissue or plant cell is defined as a growth rate or rate of cell division of the plant, plant organ, plant tissue, or plant cell that is at least 35%, at least 50%, at least 60%, or at least 75% of the growth rate or rate of cell division in a corresponding plant, plant organ, plant tissue or plant cell expressing the wild type EPSPS protein.

According to the present invention, substantially normal development of a plant, plant organ, plant tissue or plant cell is defined as the occurrence of one or more developmental events in the plant, plant organ, plant tissue or plant cell that are substantially the same as those occurring in a corresponding plant, plant organ, plant tissue or plant cell expressing the wild type EPSPS protein.

According to the present invention plant organs include, but are not limited to, leaves, stems, roots, vegetative buds, floral buds, meristems, embryos, cotyledons, endosperm, sepals, petals, pistils, carpels, stamens, anthers, microspores, pollen, pollen tubes, ovules, ovaries and fruits, or sections, slices or discs taken therefrom. Plant tissues include, but are not limited to, callus tissues, ground tissues, vascular tissues, storage tissues, meristematic tissues, leaf tissues, shoot tissues, root tissues, gall tissues, plant tumor tissues, and reproductive tissues. Plant cells include, but are not limited to, isolated cells with cell walls, variously sized aggregates thereof, and protoplasts.

Plants are substantially “tolerant” to glyphosate when they are subjected to it and provide a dose/response curve which is shifted to the right when compared with that provided by similarly subjected non-tolerant like plant. Such dose/response curves have “dose” plotted on the X-axis and “percentage kill”, “herbicidal effect”, etc., plotted on the y-axis. Tolerant plants will require more herbicide than non-tolerant like plants in order to produce a given herbicidal effect. Plants which are substantially “resistant” to the glyphosate exhibit few, if any, necrotic, lytic, chlorotic or other lesions, when subjected to glyphosate at concentrations and rates which are typically employed by the agrochemical community to kill weeds in the field. Plants which are resistant to a herbicide are also tolerant of the herbicide. The terms “resistant” and “tolerant” are to be construed as “tolerant and/or resistant” within the context of the present application.

The term “EPSPS homolog” or any variation therefore refers to an EPSPS gene or EPSPS gene product found in another plant species that performs the same or substantially the same biological function as the EPSPS genes disclosed herein and where the nucleic acid sequences or polypeptide sequences (of the EPSPS gene product) are said to be “identical” or at least 50% similar (also referred to as ‘percent identity’ or ‘substantially identical’) as described below. Two polynucleotides or polypeptides are identical if the sequence of nucleotides or amino acid residues, respectively, in the two sequences is the same when aligned for maximum correspondence as described below. The terms “identical” or “percent identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. For polypeptides where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a ‘score of zero, a conservative substitution is given a score between zero and 1. The scoring of conservative substitutions is calculated according to, e.g., the algorithm of Meyers & Miller, Computer Applic. Biol. Sci. 4: 11-17 (1988) e.g., as implemented in the program PC/GENE (Intelligenetics, Mountain View, Calif., USA).

The phrases “substantially identical,” and “percent identity” in the context of two nucleic acids or polypeptides, refer to sequences or subsequences that have at least 50%, advantageously 60%, preferably 70%, more preferably 80%, and most preferably 90-95% nucleotide or amino acid residue identity when aligned for maximum correspondence over a comparison window as measured using one of the following sequence comparison algorithms or by manual alignment and visual inspection. This definition also refers to the complement of a test sequence, which has substantial sequence or subsequence complementarity when the test sequence has substantial identity to a reference sequence.

One of skill in the art will recognize that two polypeptides can also be “substantially identical” if the two polypeptides are immunologically similar. Thus, overall protein structure may be similar while the primary structure of the two polypeptides display significant variation. Therefore a method to measure whether two polypeptides are substantially identical involves measuring the binding of monoclonal or polyclonal antibodies to each polypeptide. Two polypeptides are substantially identical if the antibodies specific for a first polypeptide bind to a second polypeptide with an affinity of at least one third of the affinity for the first polypeptide. For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, 0.4dv. Appl. Math. 2:482 (I 98 I), by the homology alignment algorithm of Needleman & Wunsch, J. Mol. Biol. 48:443 (1970), by the search for similarity method of Pearson & Lipman, Proc. Nat'I. Acad. Sci. USA 5 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), by software for alignments such as VECTOR NTI Version #6 by InforMax, Inc. MD, USA, by the procedures described in ClustalW, Thompson, J. D., Higgins, D. G. and Gibson, T. J. (1994) CLUSTALW: improving the sensitivity of progressive multiple sequence alignment through sequence weighting, position—specific gap penalties and weight matrix choice. Nucleic Acids Research, 22:4673-4680 or by visual inspection (see generally, Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 33 89-3402, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=-4, and a comparison of both strands. For amino acid sequences, the BLASTP program uses as defaults a word length (W) of 3, an expectation (E) of 10, and the BLOSUM62 scoring matrix (see Henikoff& Henikoff, Proc. Natl. Acad. Sci. USA 89:10915 (1989)). In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### 4. BRIEF DESCRIPTION OF THE FIGURES

FIG. 1 is the cDNA sequence and the amino acid sequence of Arabidopsis thaliana EPSPS gene. The underlined nucleotide and amino acid residues are the targeted residues. (GenBank accession number AY040065)

FIG. 2 shows (1) a table of the present EPSPS mutants by comparing the mutated amino acid positions in the E. Coli AroA gene product with the Arabidopsis mutations and (2) a list of (a-i) the Arabidopsis thaliana wild-type and mutant EPSPS nucleotide sequences in the region of the mutations where the upper sequence represents the wild-type sequence and the lower sequence represents the mutated sequence. The lower case nucleotides represent the mutation.

FIG. 3 is an alignment of the amino acid sequences of various EPSPS gene products performed by VECTOR NTI. The sequences were aligned using the CLUSTAL W methodology. Residues in an alignment are colored according to the following scheme:

black on window default color—non-similar residues;

blue on cyan—consensus residue derived from a block of similar residues at a given position;

black on green—consensus residue derived from the occurrence of greater than 50% of a single residue at a given position;

red on yellow—consensus residue derived from a completely conserved residue at a given position;

green on window default color—residue weakly similar to consensus residue at given position.

### 5. DETAILED DESCRIPTION OF THE INVENTION

The present invention is directed to a non-transgenic plant or plant cell having a mutation in the EPSPS gene, which plant has increased resistance or tolerance to a member of the phosphonomethylglycine family and which plant exhibits substantially normal growth or development of the plant, its organs, tissues or cells, as compared to the corresponding wild-type plant or cell. The present invention is also directed to a non-transgenic plant having a mutation in the EPSPS gene, which plant is resistant to or has an increased tolerance to a member of the phosphonomethylglycine family, e.g., glyphosate, wherein the mutated EPSPS protein has substantially the same catalytic activity as compared to the wild-type EPSPS protein.

The present invention is also directed to a method for producing a non-transgenic plant having a mutated EPSPS gene that substantially maintains the catalytic activity of the wild-type protein irrespective of the presence or absence of a herbicide of the phosphonomethylglycine family. The method comprises introducing into a plant cell a recombinagenic oligonucleobase with a targeted mutation in the EPSPS gene and identifying a cell, seed, or plant having a mutated EPSPS gene.

Illustrative examples of a recombinagenic oligonucleobase is found in following patent publications, which are incorporated in their entirety be reference herein: U.S. Pat. Nos. 5,565,350; 5,756,325; 5,871,984; 5,760,012; 5,731,181; 5,888,983; 5,795,972; 5,780,296; 5,945,339; 6,004,804; and 6,010,907 and in International Patent No. PCT/US00/23457; and in International Patent Publication Nos. WO 98/49350; WO 99/07865; WO 99/58723; WO 99/58702; and WO 99/40789.

The plant can be of any species of dicotyledonous, monocotyledonous or gymnospermous plant, including any woody plant species that grows as a tree or shrub, any herbaceous species, or any species that produces edible fruits, seeds or vegetables, or any species that produces colorful or aromatic flowers. For example, the plant may be selected from a species of plant from the group consisting of canola, sunflower, tobacco, sugar beet, cotton, maize, wheat, barley, rice, sorghum, tomato, mango, peach, apple, pear, strawberry, banana, melon, potato, sweet potato, yam, carrot, lettuce, onion, soya spp, sugar cane, pea, peanut, field beans, poplar, grape, citrus, alfalfa, rye, oats, lentils, turf and forage grasses, eucalyptus, flax, oilseed rape, cucumber, morning glory, balsam, pepper, eggplant, marigold, lotus, cabbage, daisy, carnation, tulip, iris, lily, and nut producing plants insofar as they are not already specifically mentioned.

The recombinagenic oligonucleobase can be introduced into a plant cell using any method commonly used in the art, including but not limited to, microcarriers (biolistic delivery), microfibers, electroporation, direct DNA uptake (including polyethylene mediated DNA uptake) and microinjection.

The invention is also directed to the culture of cells mutated according to the methods of the present invention in order to obtain a plant that produces seeds, henceforth a “fertile plant”, and the production of seeds and additional plants from such a fertile plant including descendant (progeny) plants that contain the mutated EPSPS gene.

The invention is further directed to a method of selectively controlling weeds in a field, the field comprising plants with the disclosed EPSPS gene alterations and weeds, the method comprising application to the field of a phosphonomethylglycine herbicide to which the said plants have been rendered resistant.

The invention is also directed to novel mutations in the EPSPS gene and gene product that confer resistance or tolerance to a member of the phosphonomethylglycine family, e.g., glyphosate, to a plant or wherein the mutated EPSPS has substantially the same enzymatic activity as compared to wild-type EPSPS.

5.1 Recombinagenic Oligonucleobases

The invention can be practiced with recombinagenic oligonucleobases having the conformations and chemistries described in U.S. Pat. No. 5,565,350 to Kmiec (Kmiec I) and U.S. Pat. No. 5,731,181 (Kmiec II) gene, which are incorporated herein by reference. Kmiec I teaches a method for introducing specific genetic alterations into a target gene. The recombinagenic oligonucleobases in Kmiec I and/or Kmiec II contain two complementary strands, one of which contains at least one segment of RNA-type nucleotides (an “RNA segment”) that are base paired to DNA-type nucleotides of the other strand.

Kmiec II discloses that purine and pyrimidine base-containing non-nucleotides can be substituted for nucleotides. U.S. Pat. Nos. 5,756,325; 5,871,984; 5,760,012; 5,888,983; 5,795,972; 5,780,296; 5,945,339; 6,004,804; and 6,010,907 and in International Patent No. PCT/US00/23457; and in International Patent Publication Nos. WO 98/49350; WO 99/07865; WO 99/58723; WO 99/58702; and WO 99/40789, which are each hereby incorporated in their entirety, disclose additional recombinagenic molecules that can be used for the present invention. The term “recombinagenic oligonucleobase” is used herein to denote the molecules that can be used in the methods of the present invention and include mixed duplex oligonucleotides, non-nucleotide containing molecules taught in Kmiec II, single stranded oligodeoxynucleotides and other recombinagenic molecules taught in the above noted patents and patent publications.

In one embodiment, the recombinagenic oligonucleobase is a mixed duplex oligonucleotide in which the RNA-type nucleotides of the mixed duplex oligonucleotide are made RNase resistant by replacing the 2′-hydroxyl with a fluoro, chloro or bromo functionality or by placing a substituent on the 2′-O. Suitable substituents include the substituents taught by the Kmiec II. Alternative substituents include the substituents taught by U.S. Pat. No. 5,334,711 (Sproat) and the substituents taught by patent publications EP 629 387 and EP 679 657 (collectively, the Martin Applications), which are incorporated herein by reference. As used herein, a 2′-fluoro, chloro or bromo derivative of a ribonucleotide or a ribonucleotide having a 2′-OH substituted with a substituent described in the Martin Applications or Sproat is termed a “2′-Substituted Ribonucleotide.” As used herein the term “RNA-type nucleotide” means a 2′-hydroxyl or 2′-Substituted Nucleotide that is linked to other nucleotides of a mixed duplex oligonucleotide by an unsubstituted phosphodiester linkage or any of the non-natural linkages taught by Kmiec I or Kmiec II. As used herein the term “deoxyribo-type nucleotide” means a nucleotide having a 2′-H, which can be linked to other nucleotides of a MDON by an unsubstituted phosphodiester linkage or any of the non-natural linkages taught by Kmiec I or Kmiec II.

In a particular embodiment of the present invention, the recombinagenic oligonucleobase is a mixed duplex oligonucleotide that is linked solely by unsubstituted phosphodiester bonds. In alternative embodiments, the linkage is by substituted phosphodiesters, phosphodiester derivatives and non-phosphorus-based linkages as taught by Kmiec II. In yet another embodiment, each RNA-type nucleotide in the mixed duplex oligonucleotide is a 2′-Substituted Nucleotide. Particularly preferred embodiments of 2′-Substituted Ribonucleotides are 2′-fluoro, 2′-methoxy, 2′-propyloxy, 2′-allyloxy, 2′-hydroxylethyloxy, 2′-methoxyethyloxy, 2′-fluoropropyloxy and 2′-trifluoropropyloxy substituted ribonucleotides. More preferred embodiments of 2′-Substituted Ribonucleotides are 2′-fluoro, 2′-methoxy, 2′-methoxyethyloxy, and 2′-allyloxy substituted nucleotides. In another embodiment the mixed duplex oligonucleotide is linked by unsubstituted phosphodiester bonds.

Although mixed duplex oligonucleotide having only a single type of 2′-substituted RNA-type nucleotide are more conveniently synthesized, the methods of the invention can be practiced with mixed duplex oligonucleotides having two or more types of RNA-type nucleotides. The function of an RNA segment may not be affected by an interruption caused by the introduction of a deoxynucleotide between two RNA-type trinucleotides, accordingly, the term RNA segment encompasses such an “interrupted RNA segment.” An uninterrupted RNA segment is termed a contiguous RNA segment. In an alternative embodiment an RNA segment can contain alternating RNase-resistant and unsubstituted 2′-OH nucleotides. The mixed duplex oligonucleotides preferably have fewer than 100 nucleotides and more preferably fewer than 85 nucleotides, but more than 50 nucleotides. The first and second strands are Watson-Crick base paired. In one embodiment the strands of the mixed duplex oligonucleotide are covalently bonded by a linker, such as a single stranded hexa, penta or tetranucleotide so that the first and second strands are segments of a single oligonucleotide chain having a single 3′ and a single 5′ end. The 3′ and 5′ ends can be protected by the addition of a “hairpin cap” whereby the 3′ and 5′ terminal nucleotides are Watson-Crick paired to adjacent nucleotides. A second hairpin cap can, additionally, be placed at the junction between the first and second strands distant from the 3′ and 5′ ends, so that the Watson-Crick pairing between the first and second strands is stabilized.

The first and second strands contain two regions that are homologous with two fragments of the target EPSPS gene, i.e., have the same sequence as the target gene. A homologous region contains the nucleotides of an RNA segment and may contain one or more DNA-type nucleotides of connecting DNA segment and may also contain DNA-type nucleotides that are not within the intervening DNA segment. The two regions of homology are separated by, and each is adjacent to, a region having a sequence that differs from the sequence of the target gene, termed a “heterologous region.” The heterologous region can contain one, two or three mismatched nucleotides. The mismatched nucleotides can be contiguous or alternatively can be separated by one or two nucleotides that are homologous with the target gene. Alternatively, the heterologous region can also contain an insertion or one, two, three or of five or fewer nucleotides. Alternatively, the sequence of the mixed duplex oligonucleotide may differ from the sequence of the target gene only by the deletion of one, two, three, or five or fewer nucleotides from the mixed duplex oligonucleotide. The length and position of the heterologous region is, in this case, deemed to be the length of the deletion, even though no nucleotides of the mixed duplex oligonucleotide are within the heterologous region. The distance between the fragments of the target gene that are complementary to the two homologous regions is identically the length of the heterologous region when a substitution or substitutions is intended. When the heterologous region contains an insertion, the homologous regions are thereby separated in the mixed duplex oligonucleotide farther than their complementary homologous fragments are in the gene, and the converse is applicable when the heterologous region encodes a deletion.

The RNA segments of the mixed duplex oligonucleotides are each a part of a homologous region, i.e., a region that is identical in sequence to a fragment of the target gene, which segments together preferably contain at least 13 RNA-type nucleotides and preferably from 16 to 25 RNA-type nucleotides or yet more preferably 18-22 RNA-type nucleotides or most preferably 20 nucleotides. In one embodiment, RNA segments of the homology regions are separated by and adjacent to, i.e., “connected by” an intervening DNA segment. In one embodiment, each nucleotide of the heterologous region is a nucleotide of the intervening DNA segment. An intervening DNA segment that contains the heterologous region of a mixed duplex oligonucleotide is termed a “mutator segment.”

The change to be introduced into the target EPSPS gene is encoded by the heterologous region. The change to be introduced into the EPSPS gene may be a change in one or more bases of the EPSPS gene sequence or the addition or deletion of one or more bases.

In another embodiment of the present invention, the recombinagenic oligonucleobase is a single stranded oligodeoxynucleotide mutational vector or SSOMV, which is disclosed in International Patent Application PCT/US00/23457, which is incorporated herein by reference in its entirety. The sequence of the SSOMV is based on the same principles as the mutational vectors described in U.S. Pat. Nos. 5,756,325; 5,871,984; 5,760,012; 5,888,983; 5,795,972; 5,780,296; 5,945,339; 6,004,804; and 6,010,907 and in International Publication Nos. WO 98/49350; WO 99/07865; WO 99/58723; WO 99/58702; and WO 99/40789. The sequence of the SSOMV contains two regions that are homologous with the target sequence separated by a region that contains the desired genetic alteration termed the mutator region. The mutator region can have a sequence that is the same length as the sequence that separates the homologous regions in the target sequence, but having a different sequence. Such a mutator region can cause a substitution. Alternatively, the homolgous regions in the SSOMV can be contiguous to each other, while the regions in the target gene having the same sequence are separated by one, two or more nucleotides. Such a SSOMV causes a deletion from the target gene of the nucleotides that are absent from the SSOMV. Lastly, the sequence of the target gene that is identical to the homologous regions may be adjacent in the target gene but separated by one two or more nucleotides in the sequence of the SSOMV. Such an SSOMV causes an insertion in the sequence of target gene.

The nucleotides of the SSOMV are deoxyribonucleotides that are linked by unmodified phosphodiester bonds except that the 3′ terminal and/or 5′ terminal internucleotide linkage or alternatively the two 3′ terminal and/or 5′ terminal internucleotide linkages can be a phosphorothioate or phosphoamidate. As used herein an internucleotide linkage is the linkage between nucleotides of the SSOMV and does not include the linkage between the 3′ end nucleotide or 5′ end nucleotide and a blocking substituent, see supra. In a specific embodiment the length of the SSOMV is between 21 and 55 deoxynucleotides and the lengths of the homology regions are, accordingly, a total length of at least 20 deoxynucleotides and at least two homology regions should each have lengths of at least 8 deoxynucleotides.

The SSOMV can be designed to be complementary to either the coding or the non-coding strand of the target gene. When the desired mutation is a substitution of a single base, it is preferred that both the mutator nucleotide be a pyrimidine. To the extent that is consistent with achieving the desired functional result it is preferred that both the mutator nucleotide and the targeted nucleotide in the complementary strand be pyrimidines. Particularly preferred are SSOMV that encode transversion mutations, i.e., a C or T mutator nucleotide is mismatched, respectively, with a C or T nucleotide in the complementary strand.

In addition to the oligodeoxynucleotide the SSOMV can contain a 5′ blocking substituent that is attached to the 5′ terminal carbons through a linker. The chemistry of the linker is not critical other than its length, which should preferably be at least 6 atoms long and that the linker should be flexible. A variety of non-toxic substituents such as biotin, cholesterol or other steroids or a non-intercalating cationic fluorescent dye can be used. Particularly preferred as reagents to make SSOMV are the reagents sold as Cy3™ and Cy5™ by Glen Research, Sterling VA, which are blocked phosphoroamidites that upon incorporation into an oligonucleotide yield 3,3,3′,3′-tetramethyl N,N′-isopropyl substituted indomonocarbocyanine and indodicarbocyanine dyes, respectively. Cy3 is the most preferred. When the indocarbocyanine is N-oxyalkyl substituted it can be conveniently linked to the 5′ terminal of the oligodeoxynucleotide through as a phosphodiester with a 5′ terminal phosphate. The chemistry of the dye linker between the dye and the oligodeoxynucleotide is not critical and is chosen for synthetic convenience. When the commercially available Cy3 phosphoramidite is used as directed the resulting 5′ modification consists of a blocking substituent and linker together which are a N-hydroxypropyl, N′-phosphatidylpropyl 3,3,3′,3′-tetramethyl indomonocarbocyanine.

In the preferred embodiment the indocarbocyanine dye is tetra substituted at the 3 and 3′ positions of the indole rings. Without limitation as to theory these substitutions prevent the dye from being an intercalating dye. The identity of the substituents at these positions are not critical. The SSOMV can in addition have a 3′ blocking substituent. Again the chemistry of the 3′ blocking substituent is not critical.

5.2 The Location and Type of Mutation Introduced into the EPSPS Gene

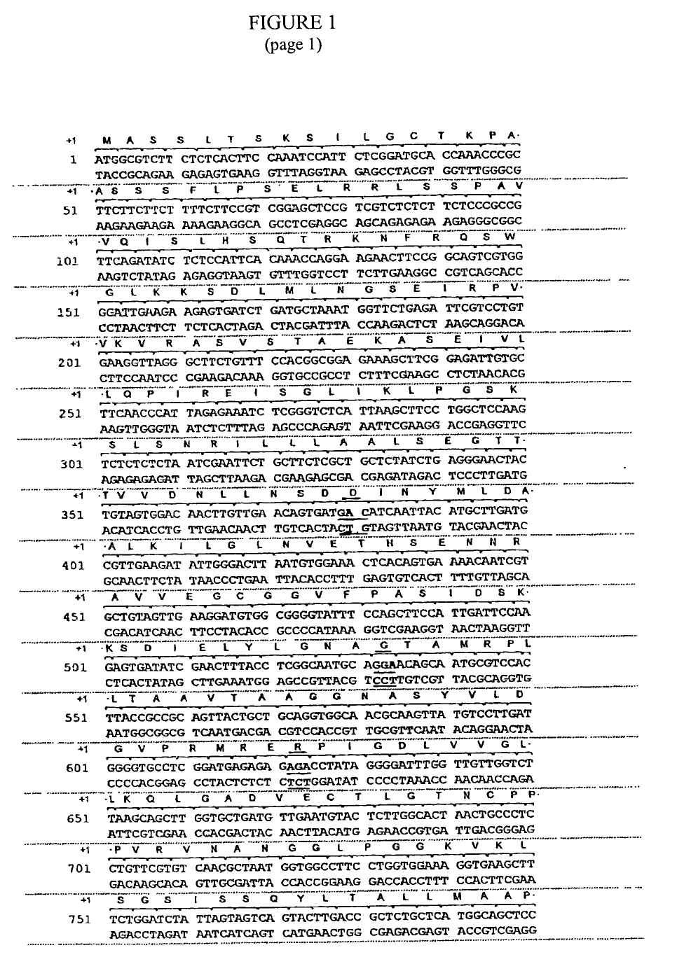

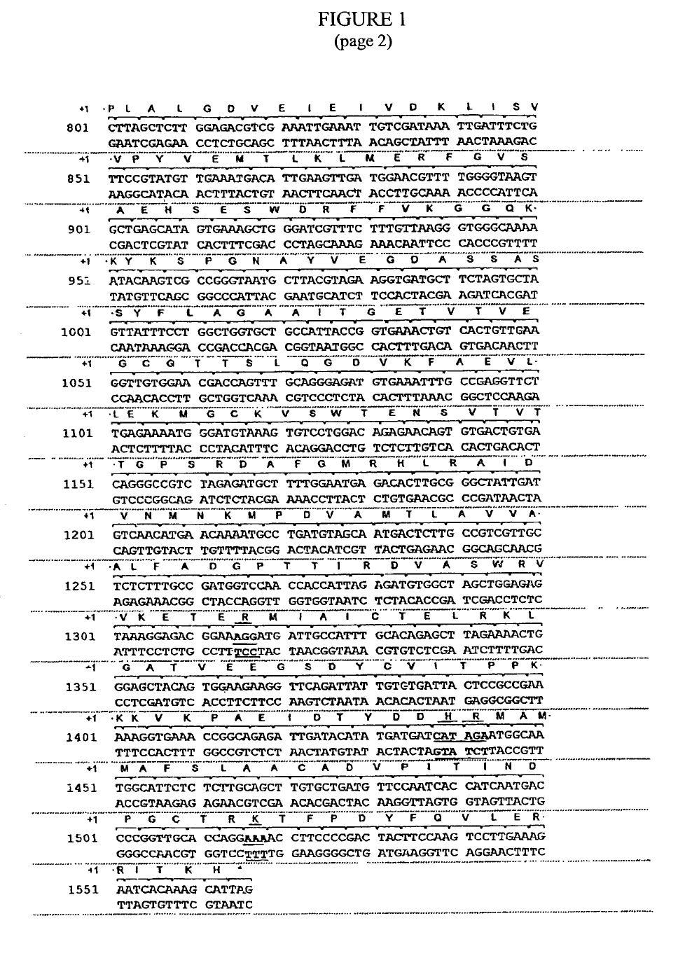

In one embodiment of the present invention, the Arabidopsis thaliana EPSPS gene and corresponding EPSPS gene product (enzyme) (see FIG. 1) comprises a mutation at one or more amino acid residues selected from the group consisting of D₁₂₆, R₂₀₇, R438, H₄₇₉, R480, G₁₇₇ and K₅₀₅ or at an analogous position in an EPSPS homolog, and the mutation results in one or more of the following amino acid substitutions in the EPSPS enzyme in comparison with the wild-type sequence:

(i) Asp₁₂₆—Glu

(ii) Arg207—Glu

(iii) Arg438—Lys

(iv) His₄₇₉—Arg or Leu

(v) His₄₇₉R₄₈₀—Arg₄₇₉Lys₄₈₀

(vi) Gly₁₇₇—Met or Ser

(vii) Lys₅₀₅—Arg

Alternatively, and/or additionally, the mutation may result in the replacement of any amino acid at positions corresponding to 126, 177, 207, 438, 479, 480 (if amino acid 479 is replaced) and 505 with respect to the EPSPS protein depicted in FIG. 1.

In specific embodiments of the present invention, the EPSPS gene is mutated at amino acid position 126 in which Asp is replaced by Glu. Another specific embodiment is the substitution of Arg at amino acid position 207 by Glu. A further specific embodiment comprises a mutation at amino acid position 480 in which Arg is replaced by Lys, plus the additional substitution of His at amino acid position 479 by Arg. Other specific embodiments of the present invention are directed to mutations at amino acid position 438, in which Arg is replaced by Lys; amino acid position 479, in which His is replaced by Arg or Leu; amino acid position 177 in which Gly is substituted by Ser or Met; and amino acid position 505 in which Lys is replaced by Arg.

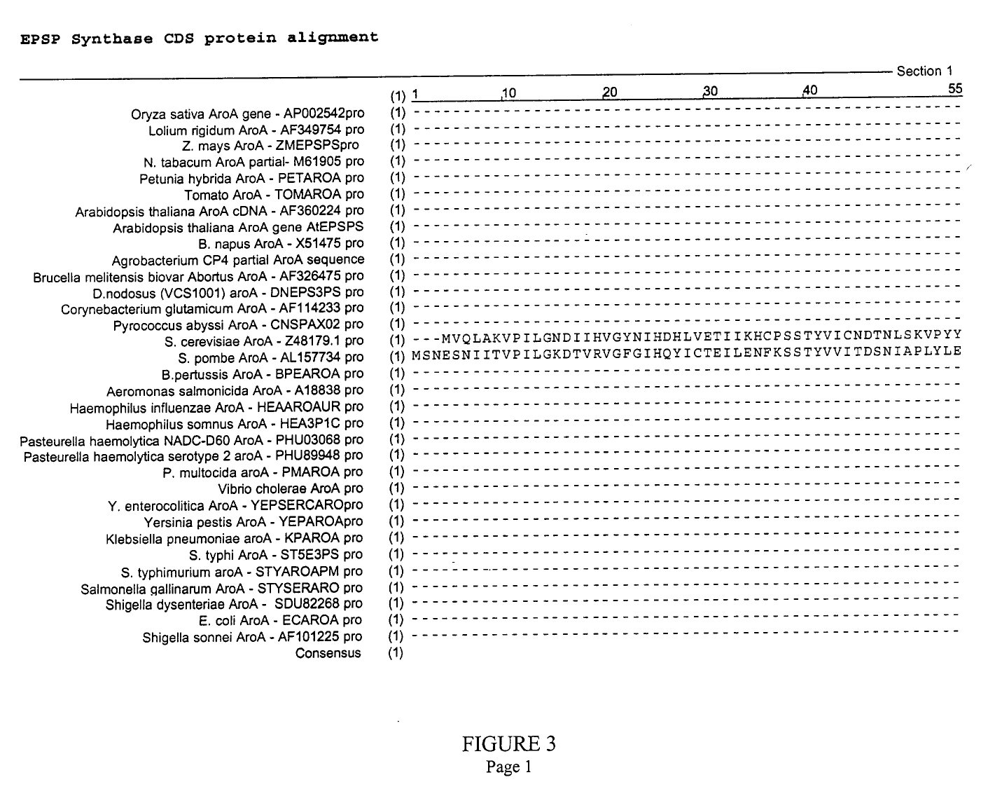

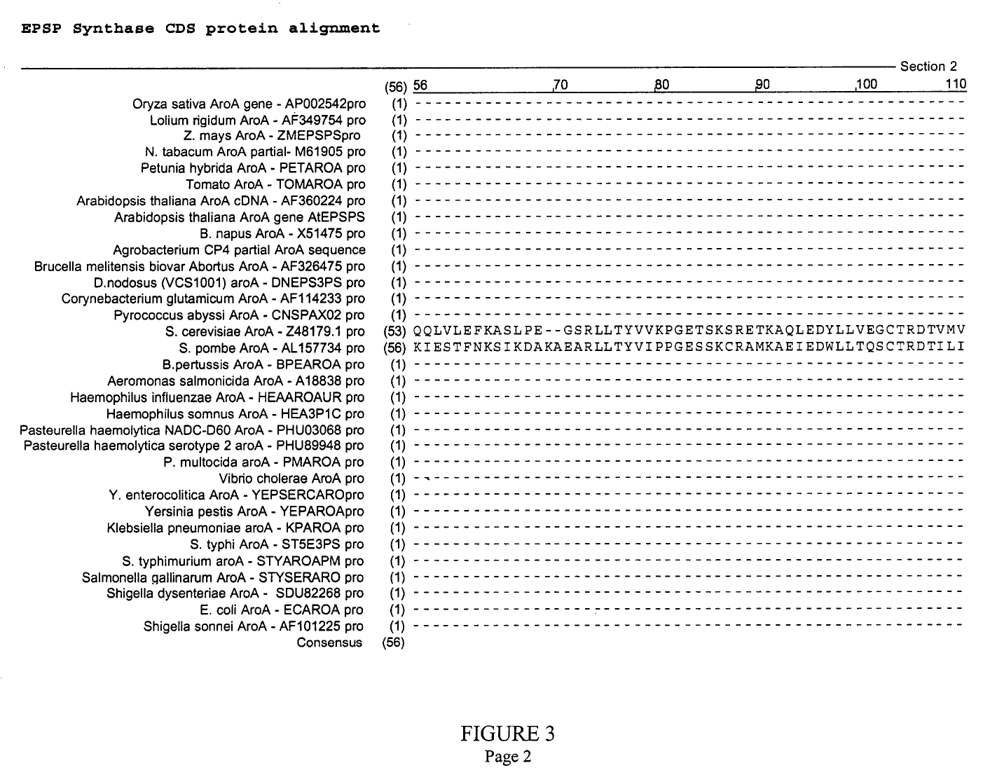

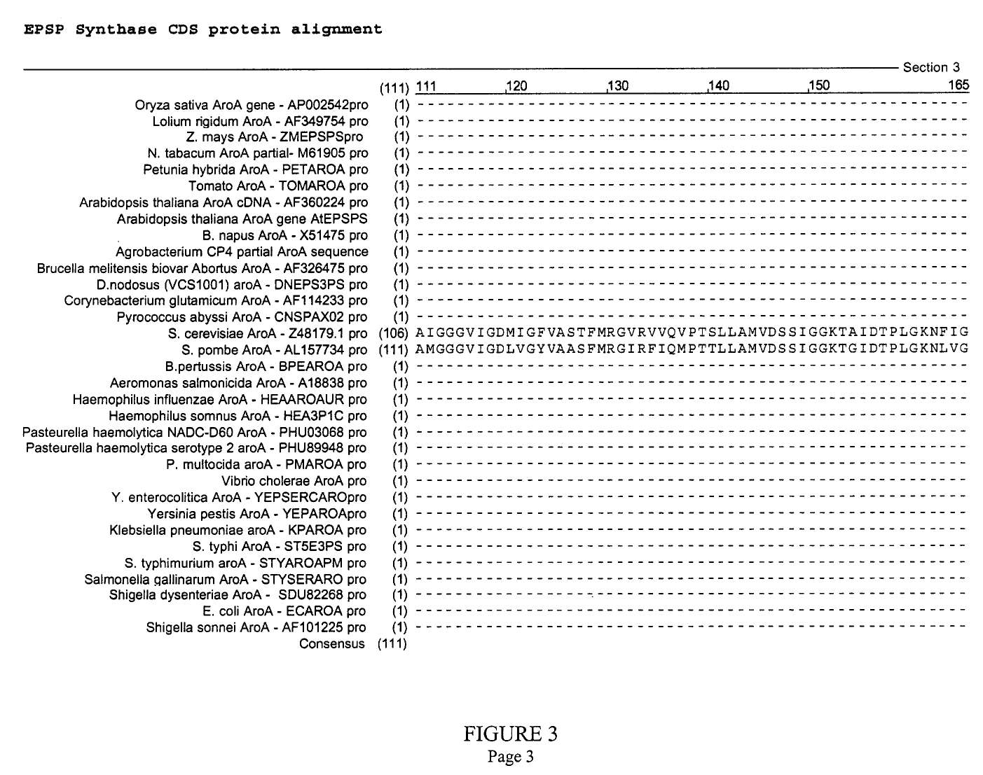

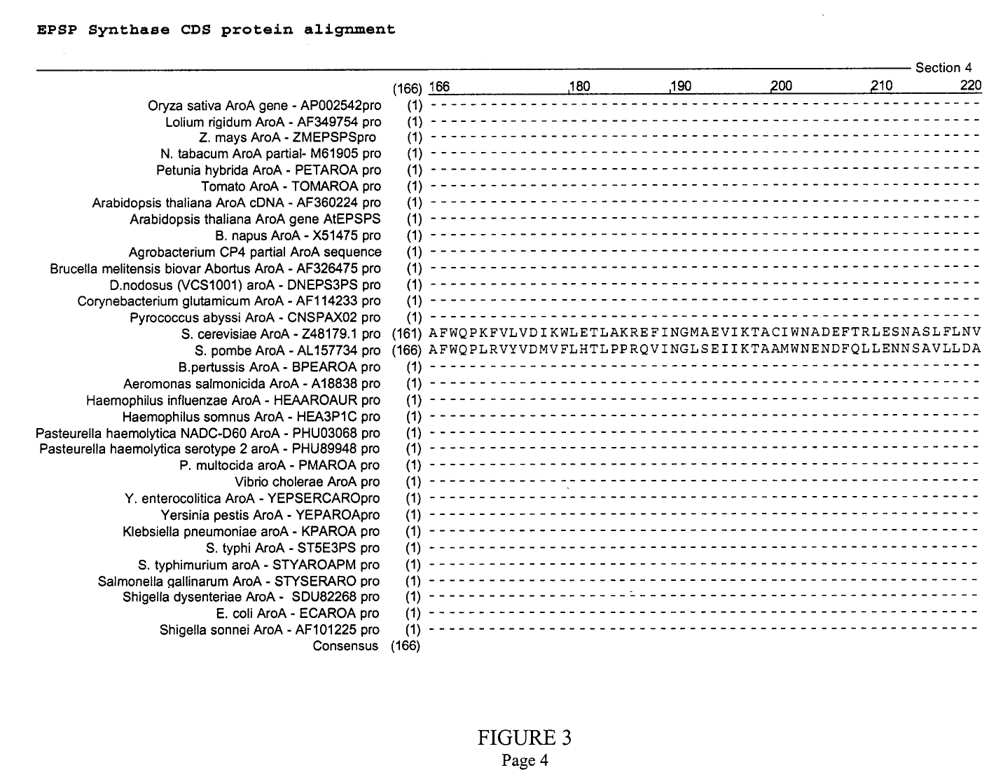

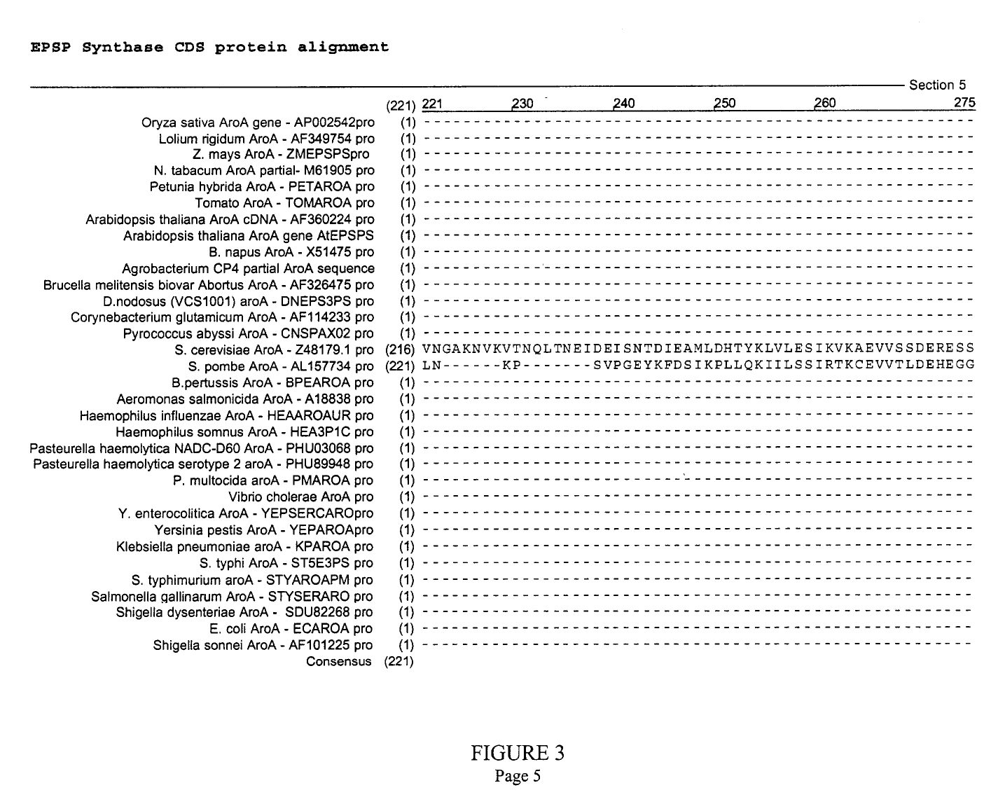

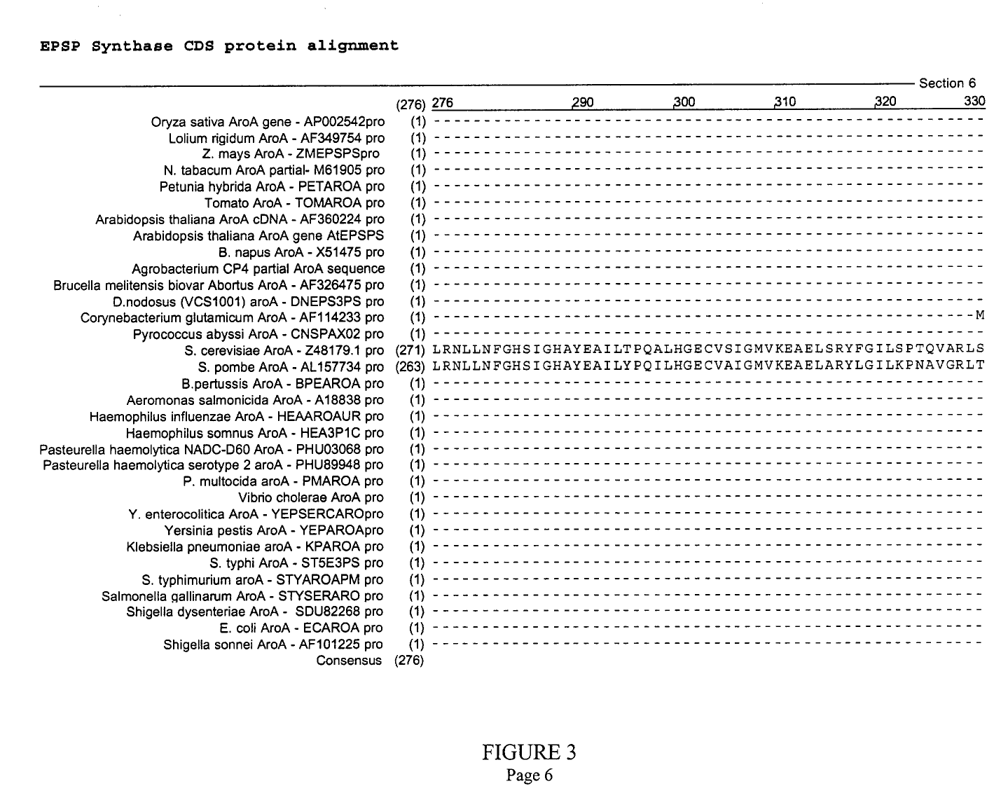

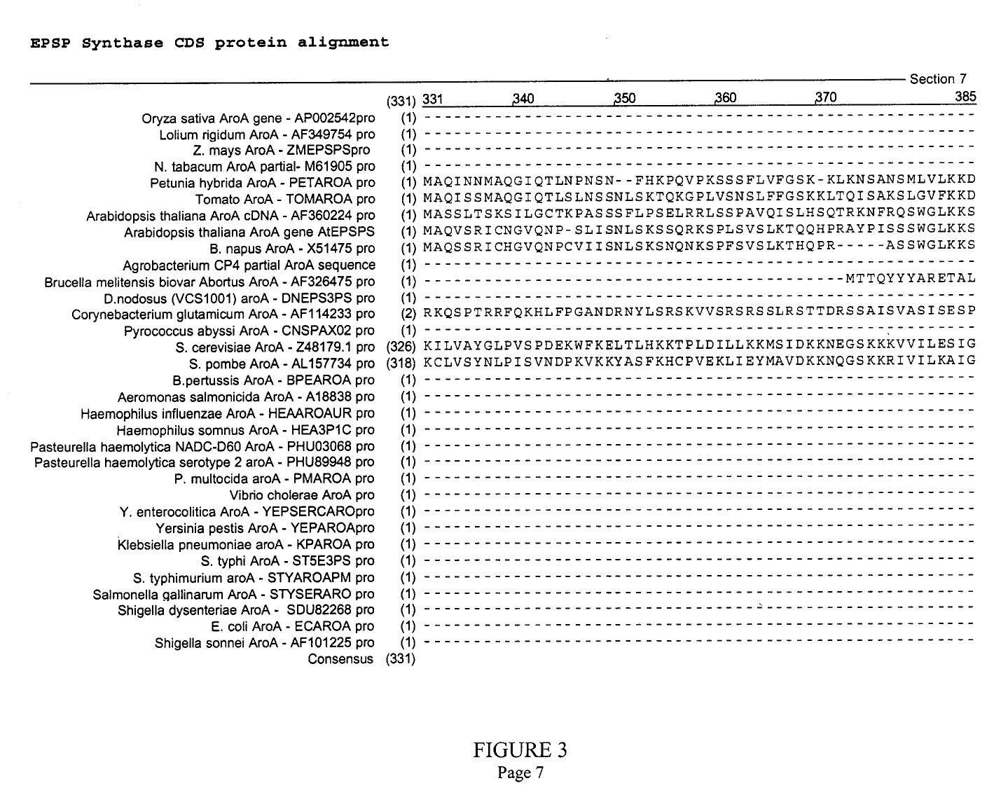

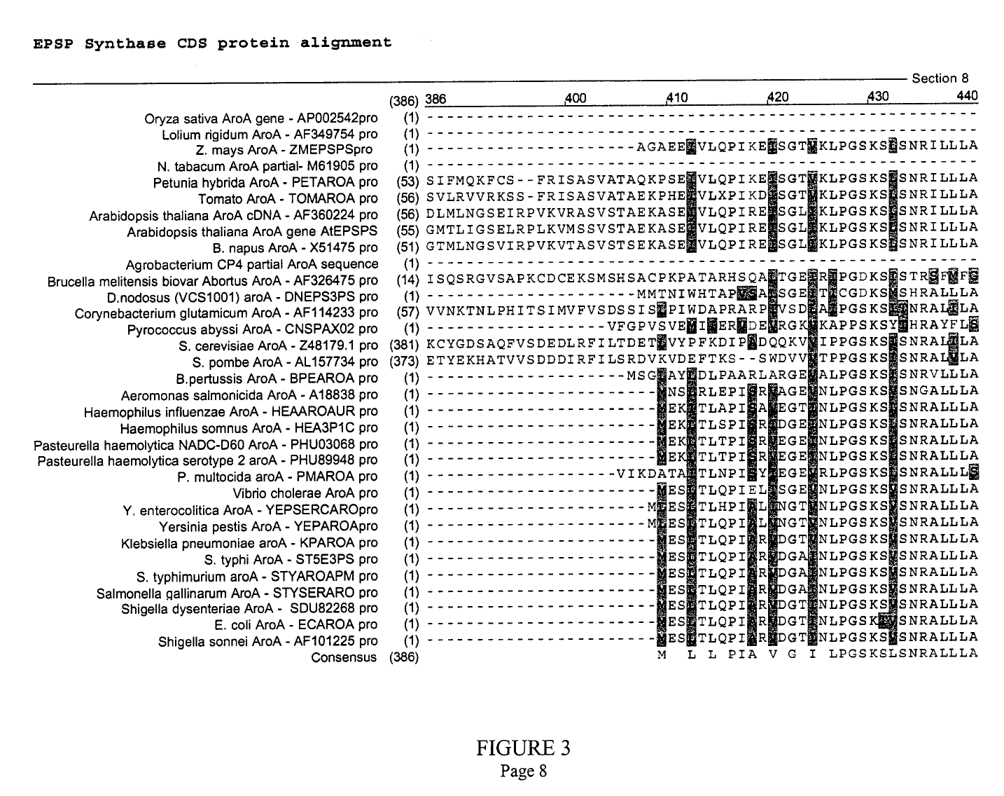

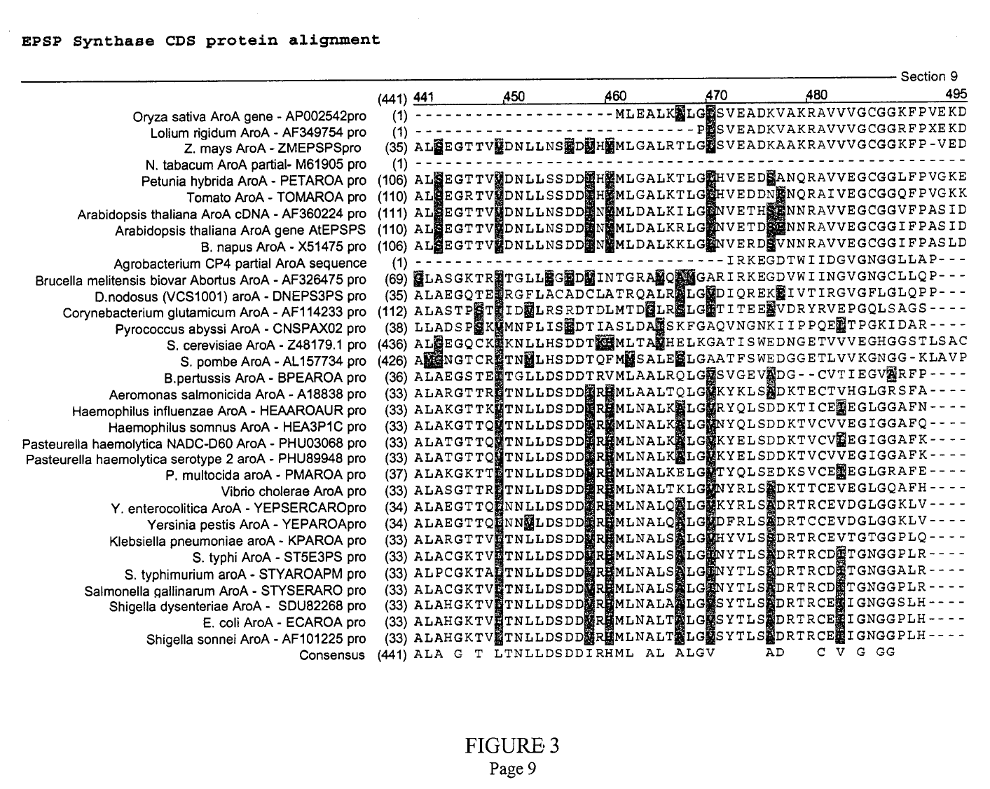

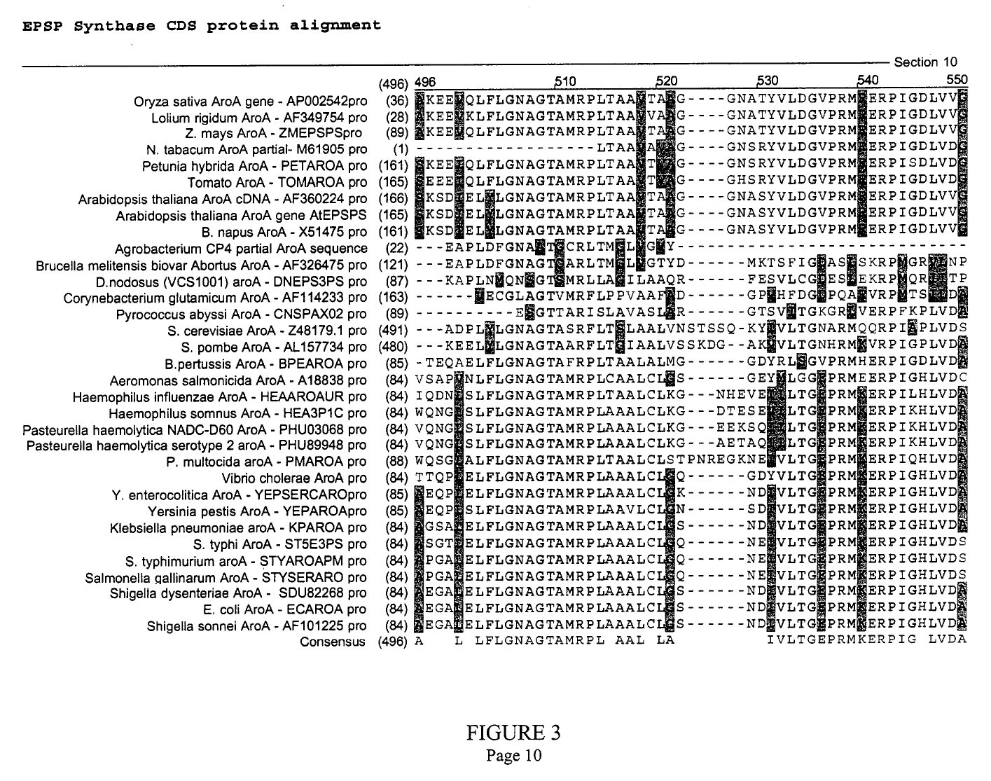

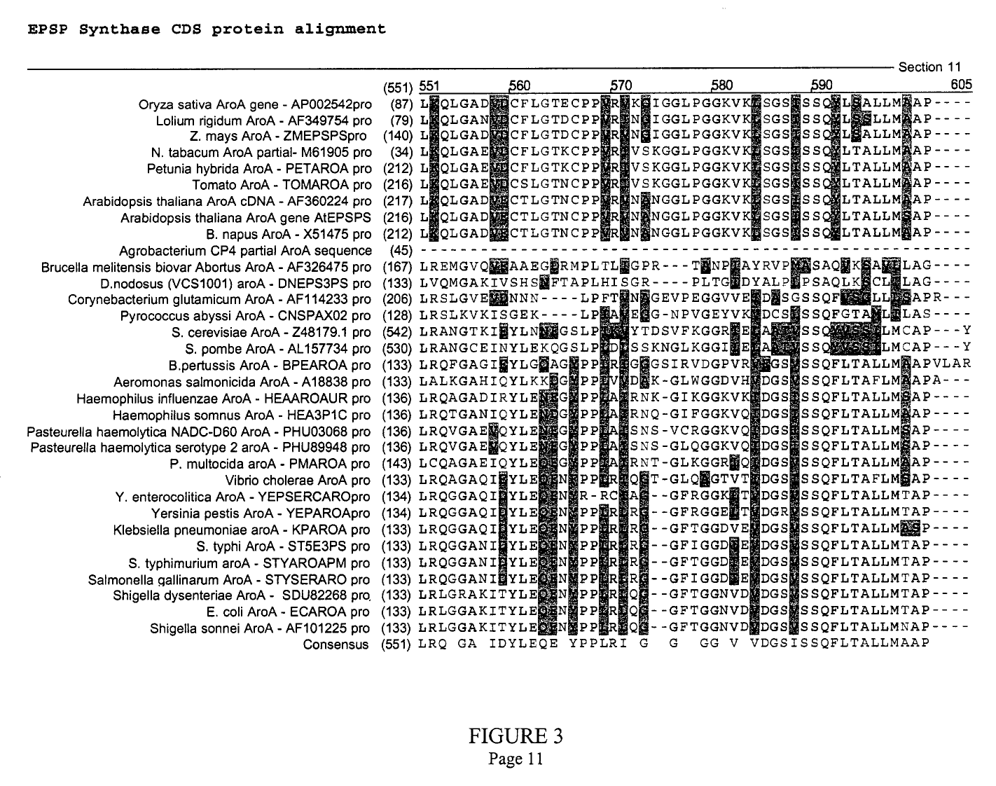

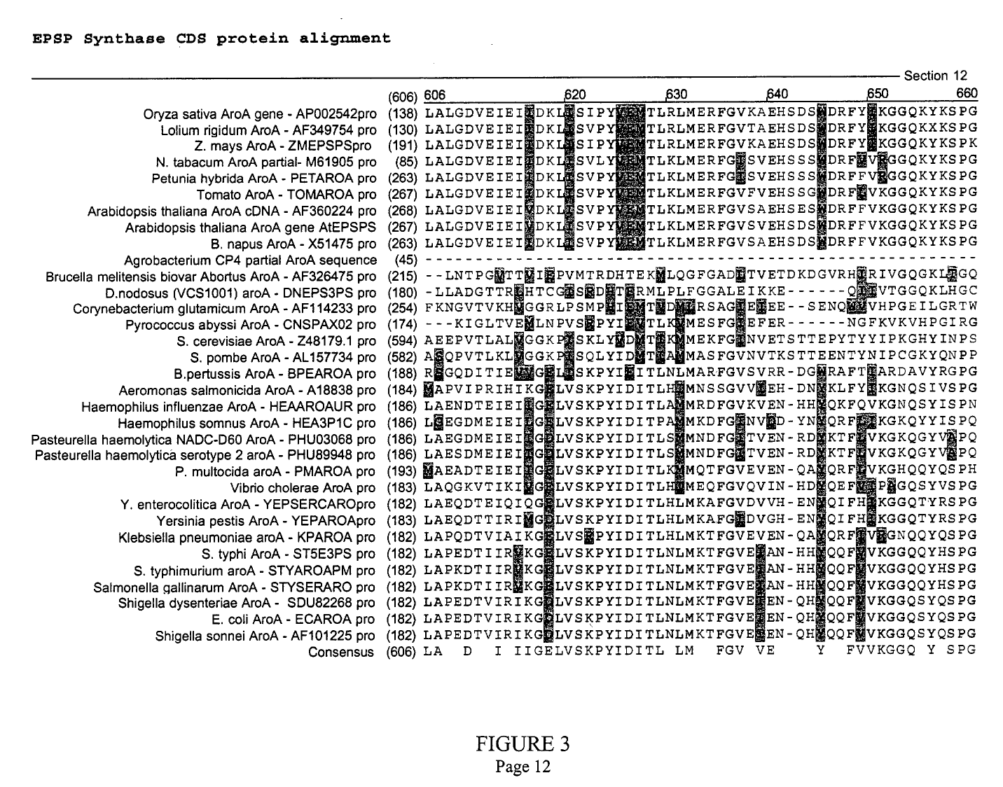

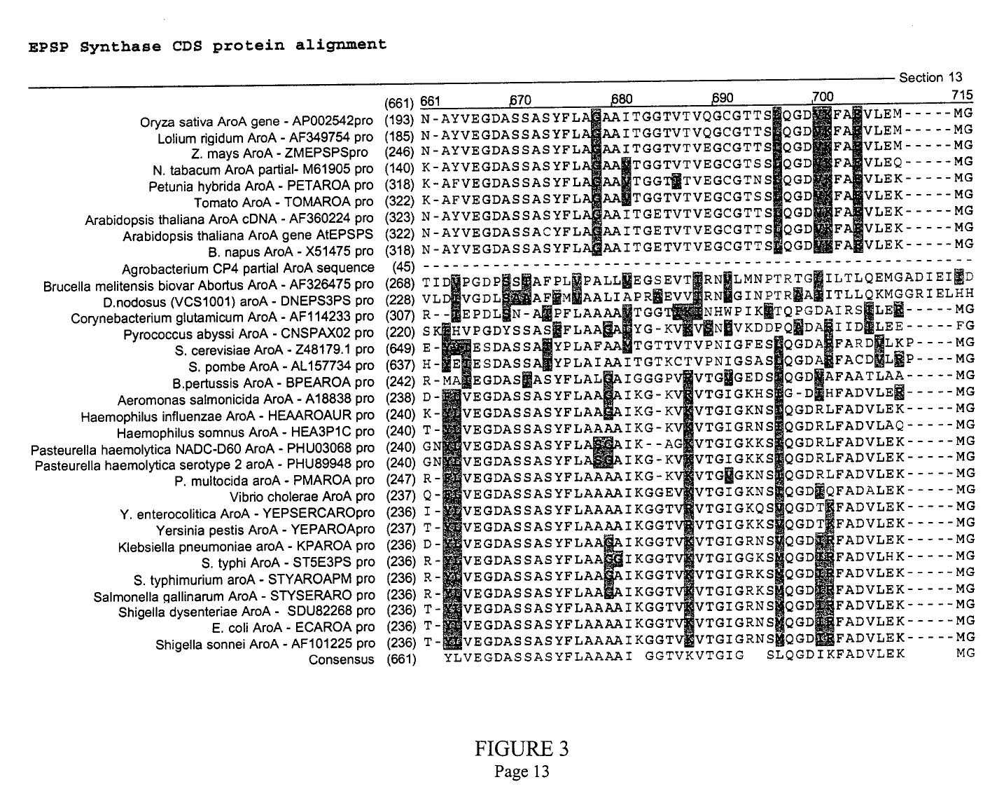

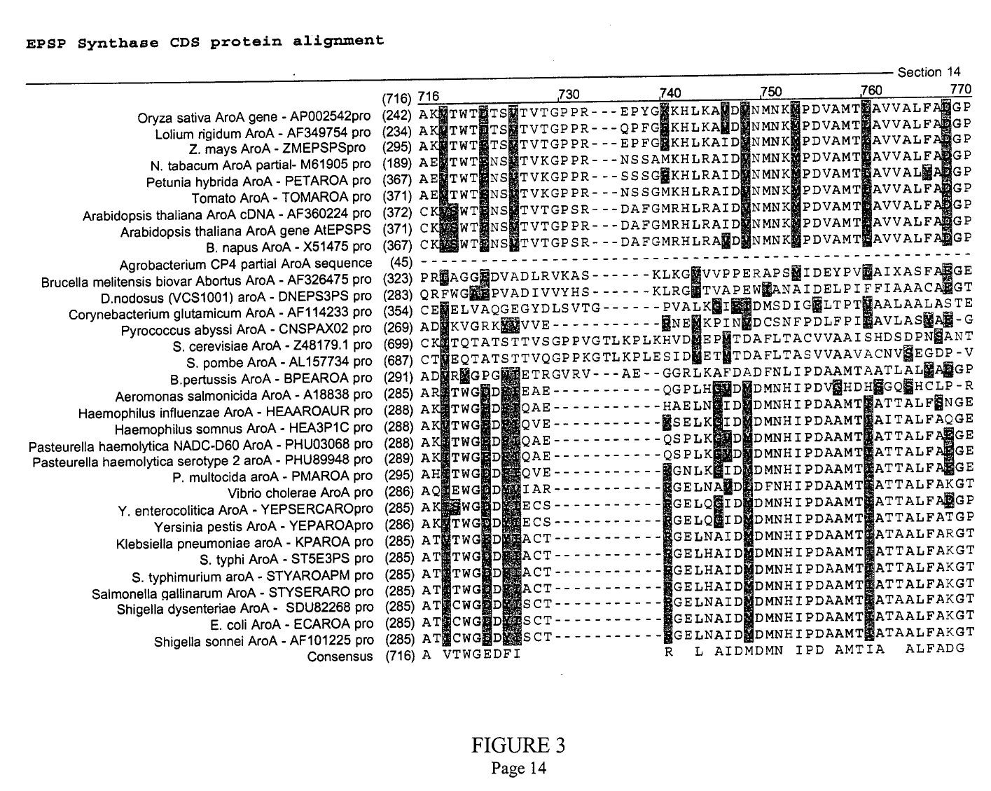

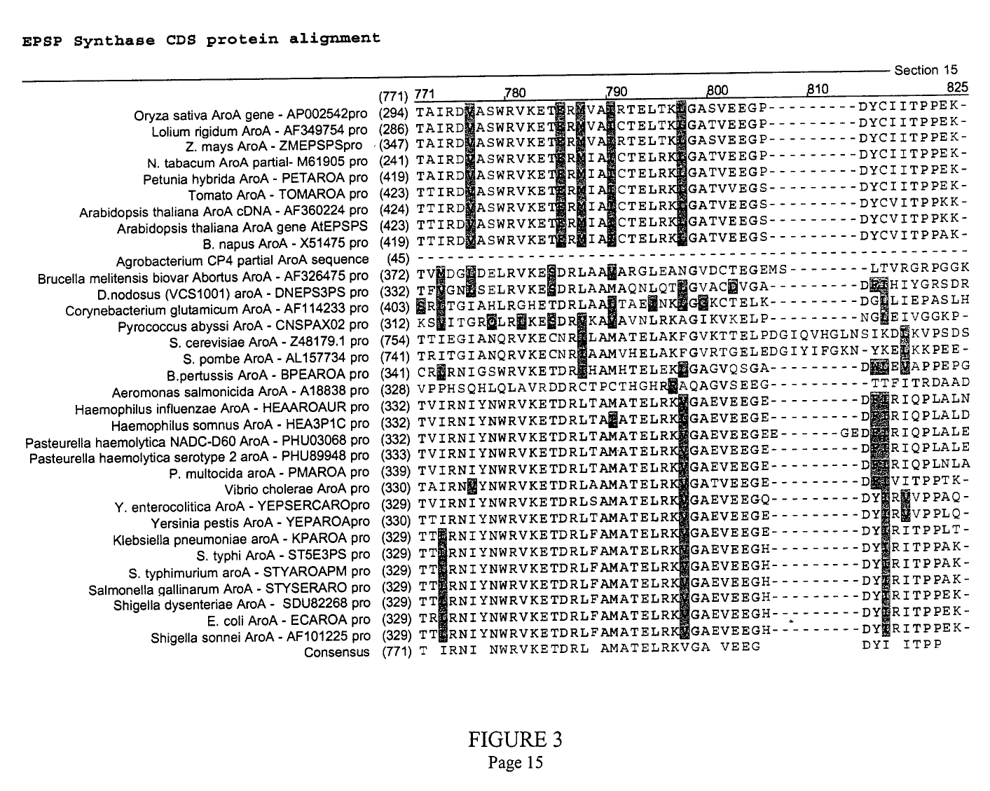

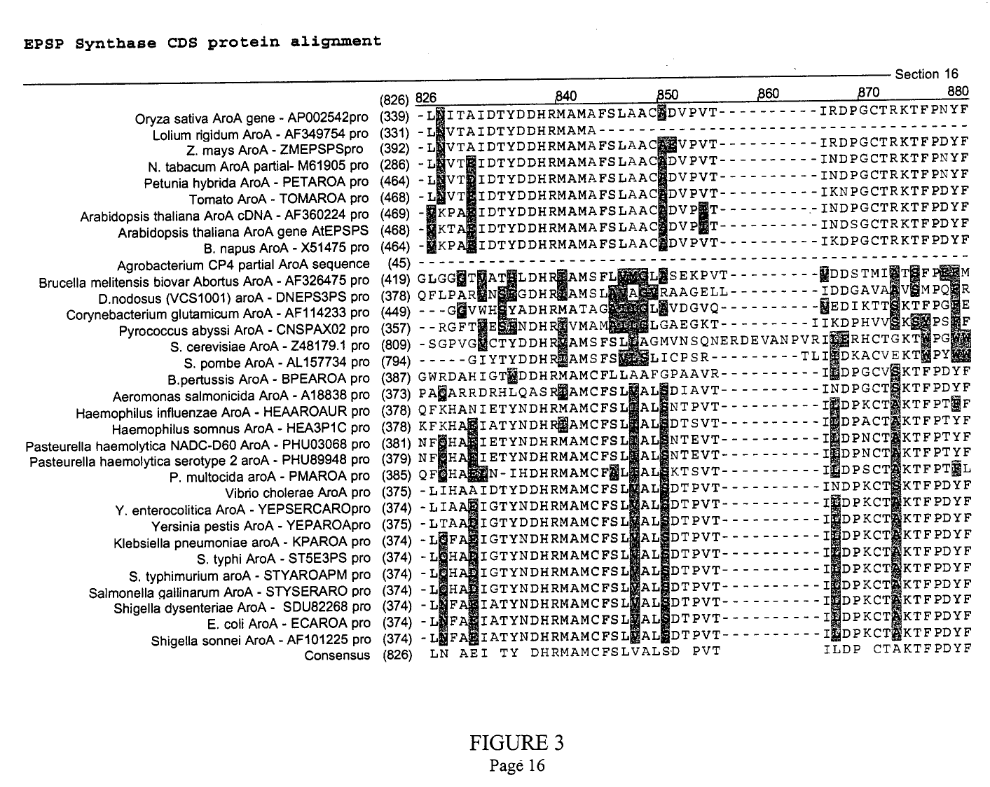

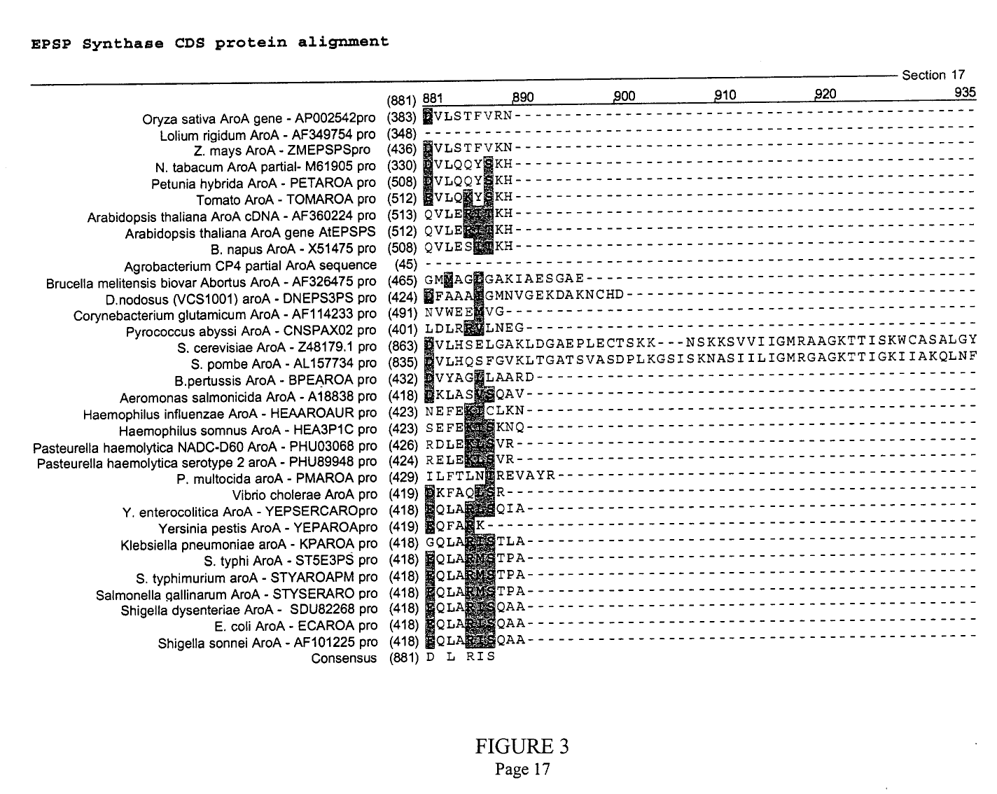

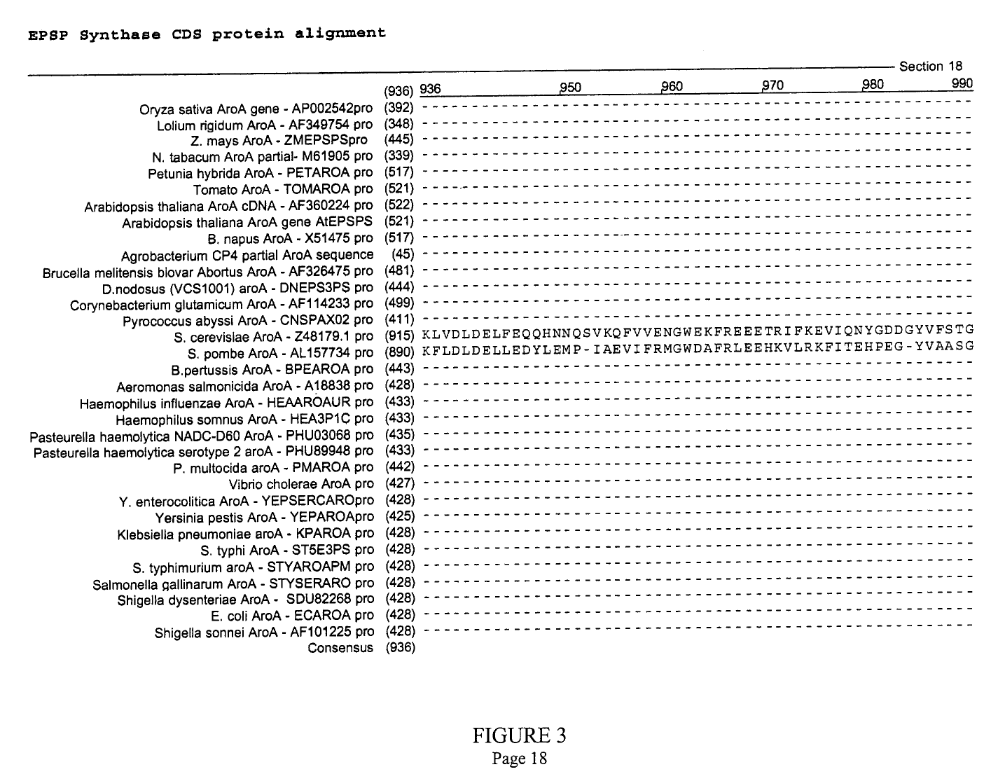

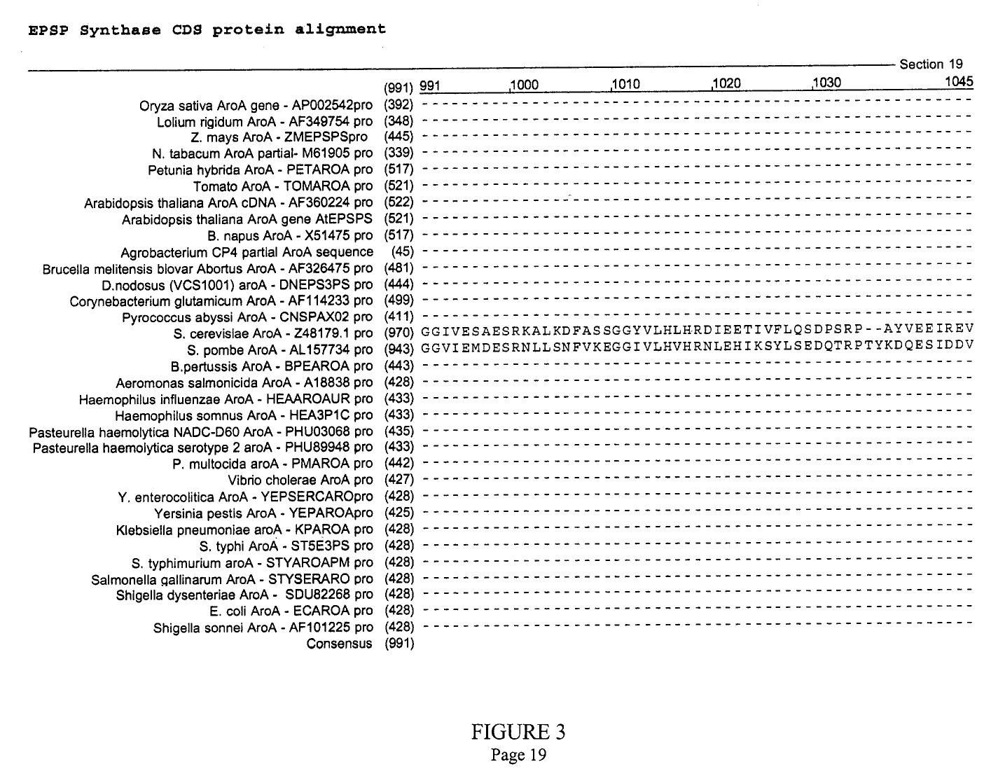

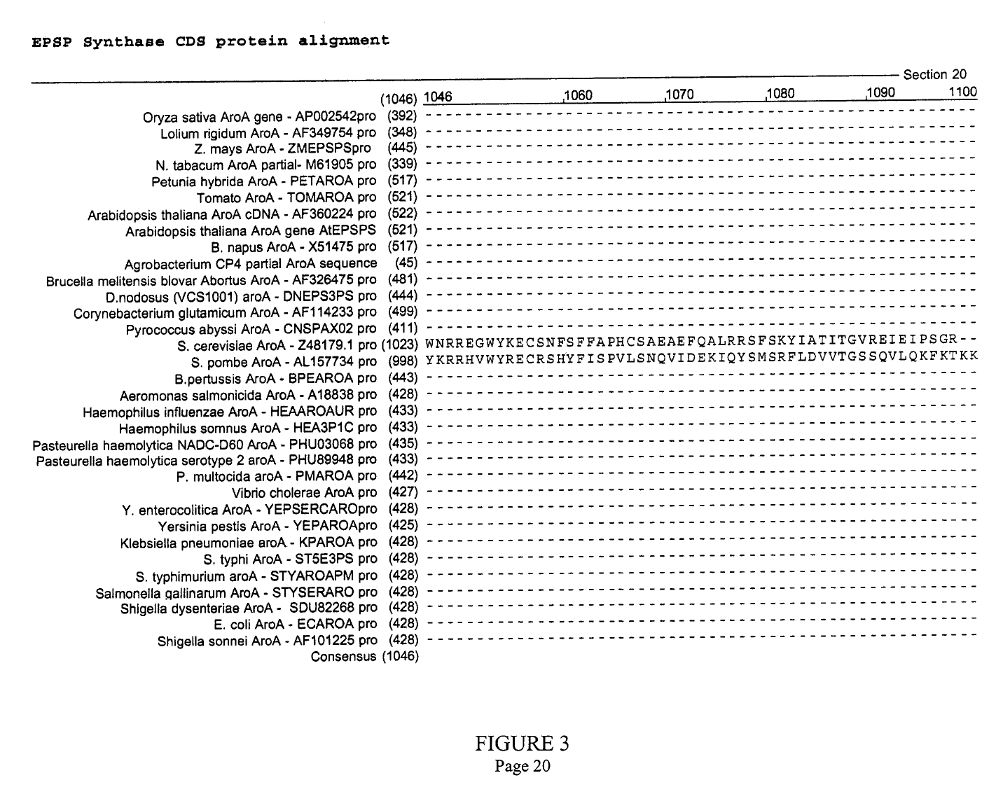

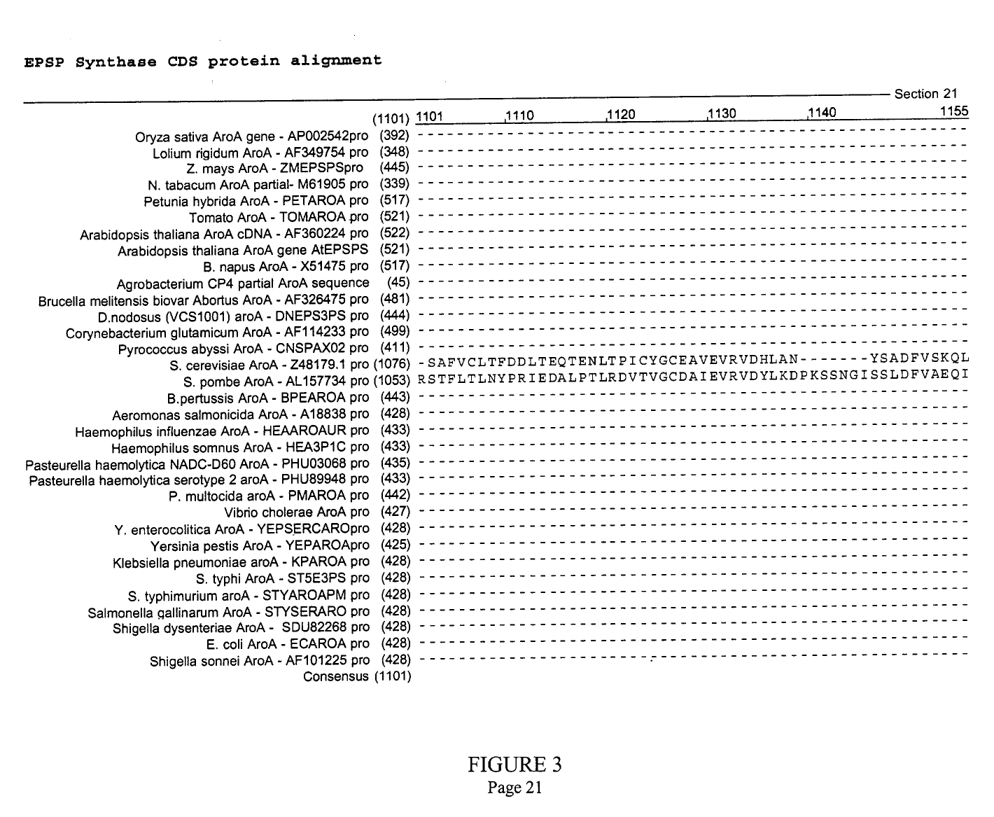

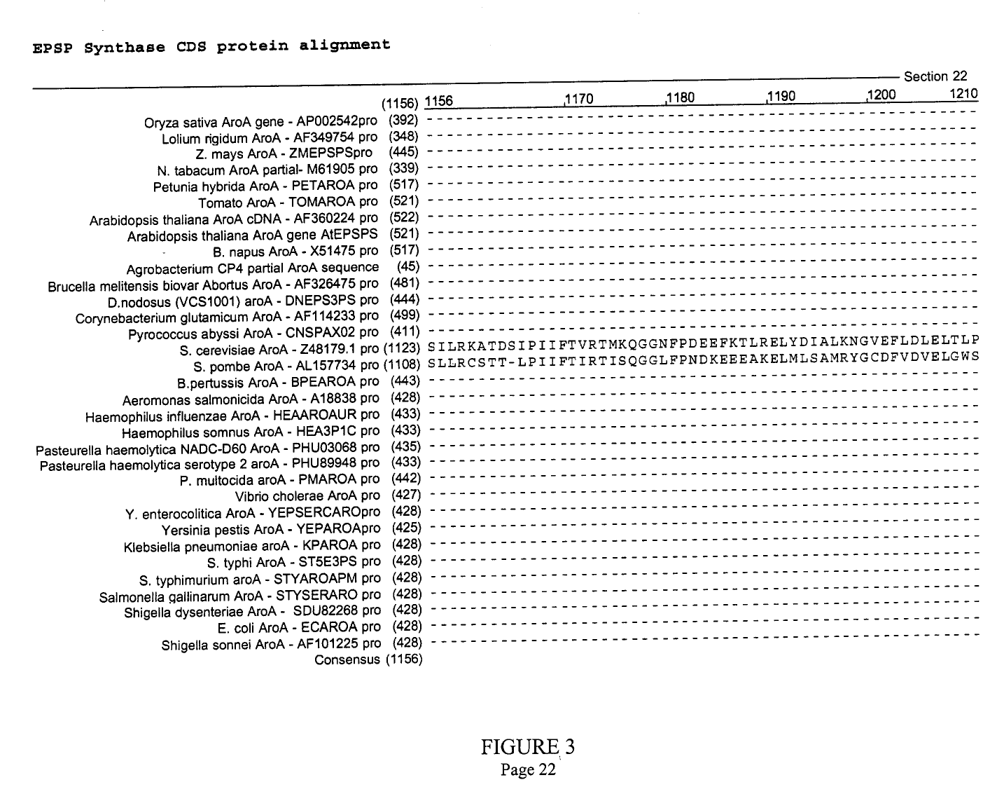

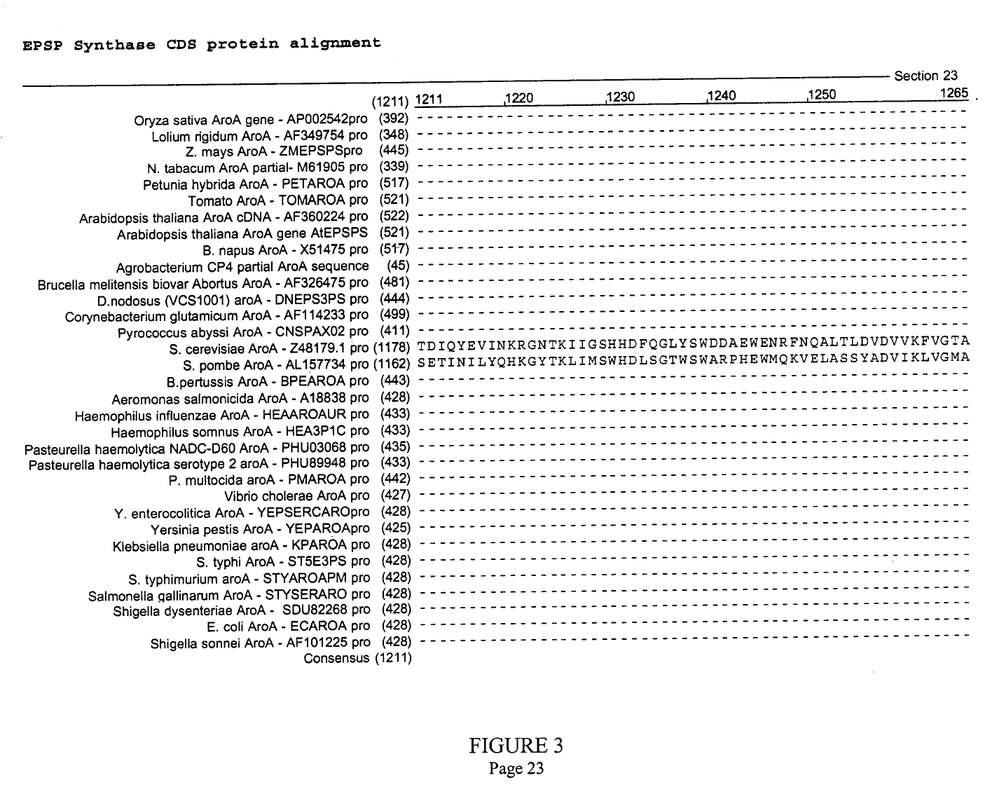

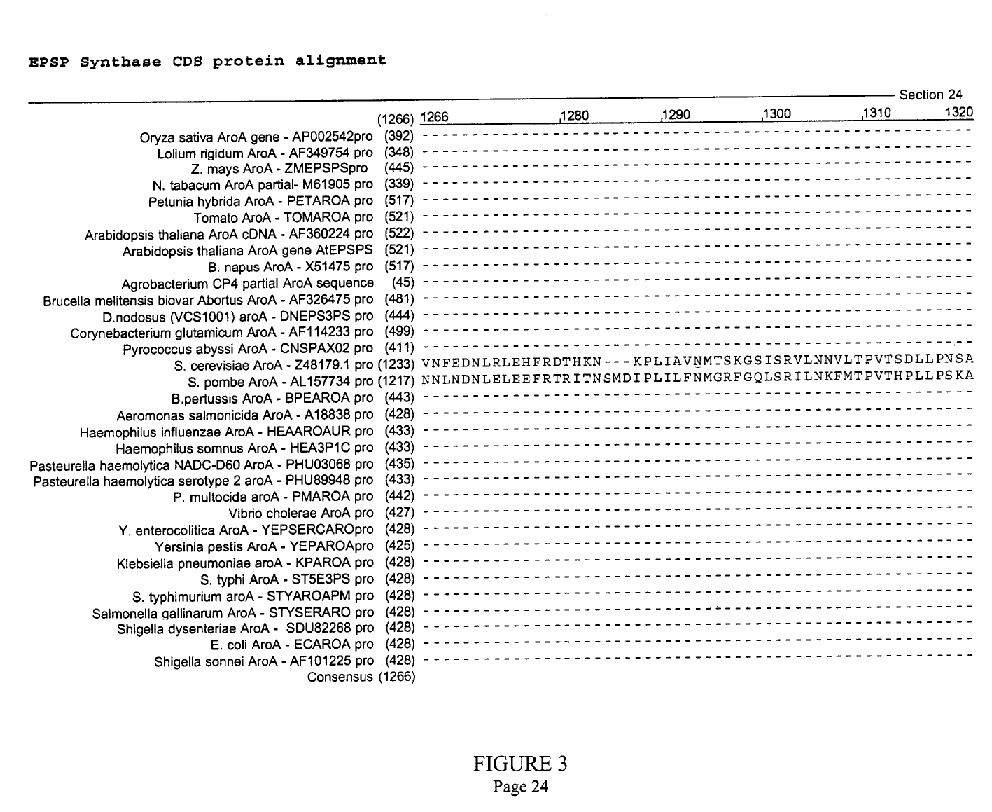

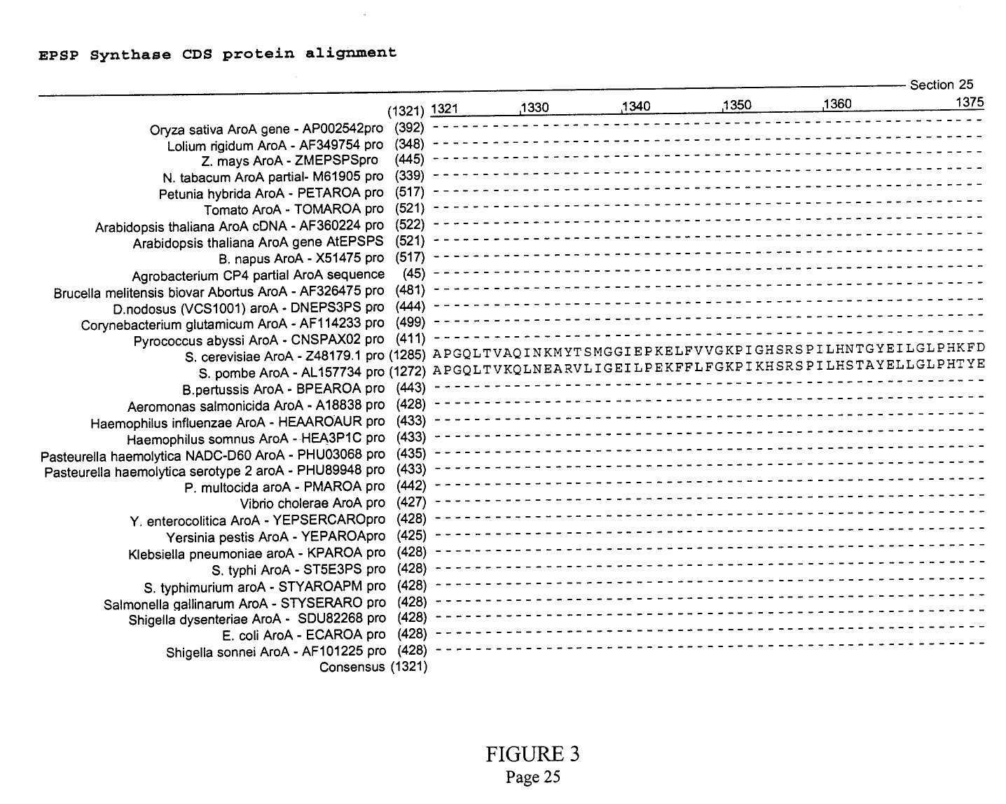

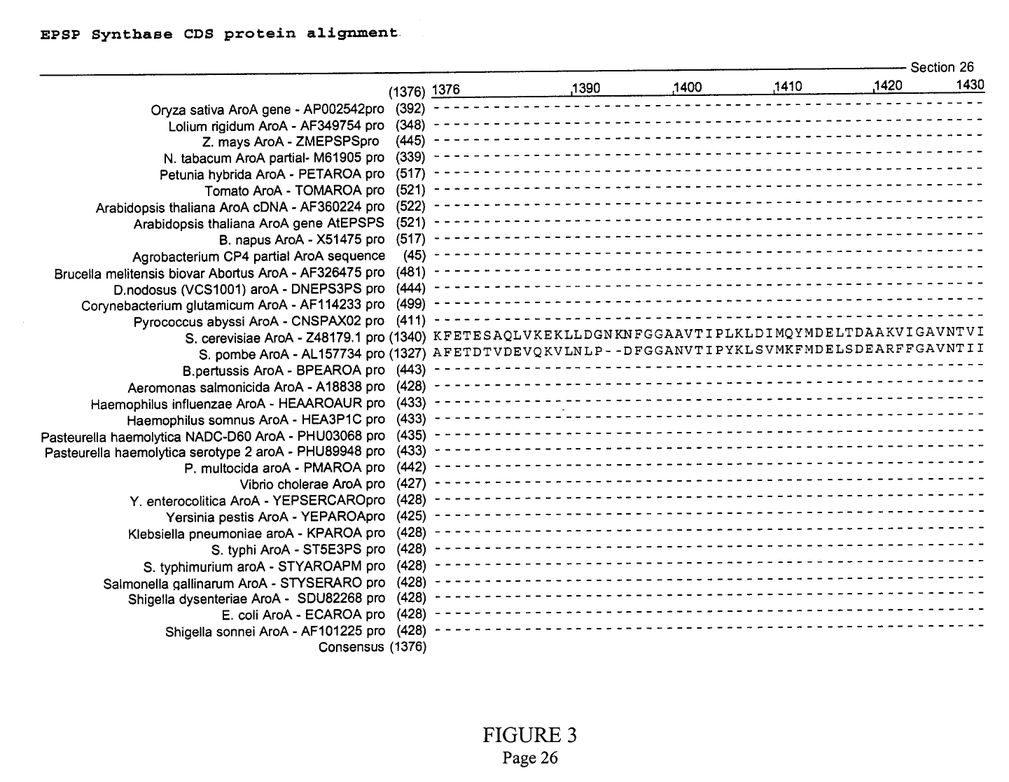

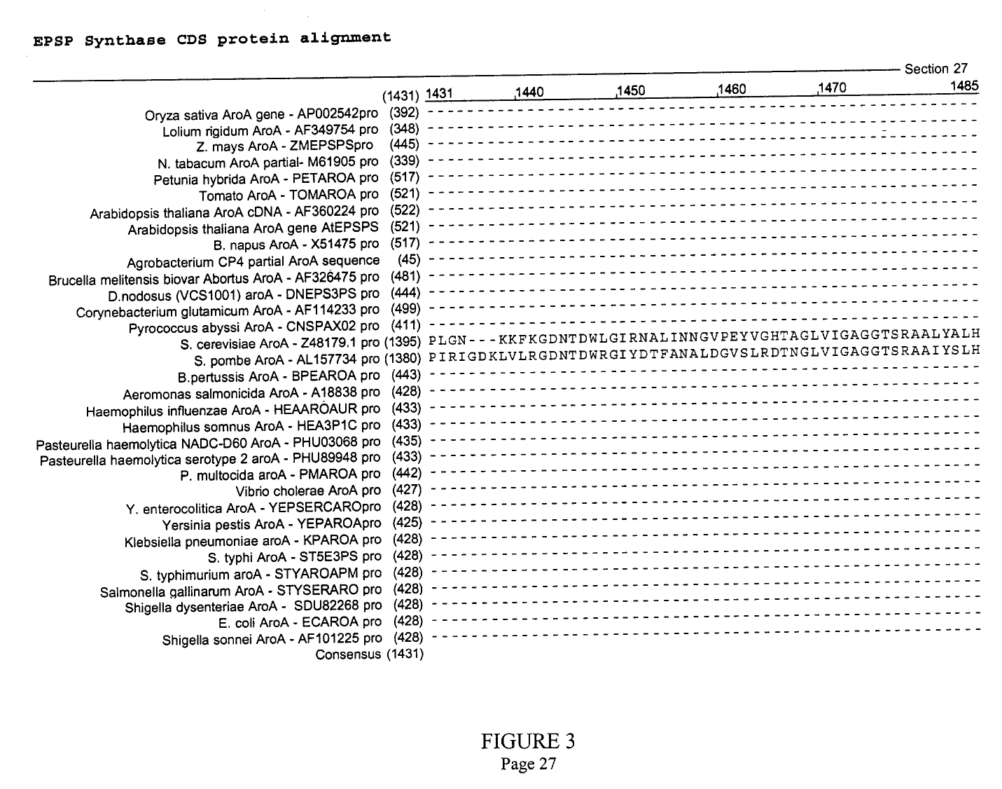

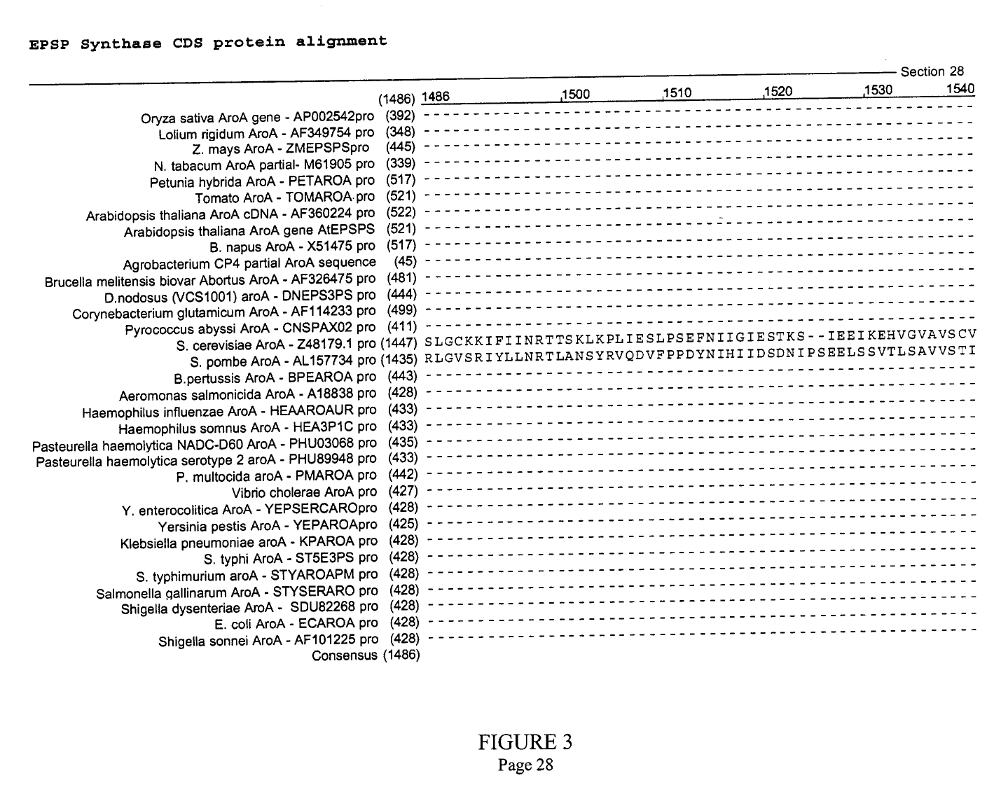

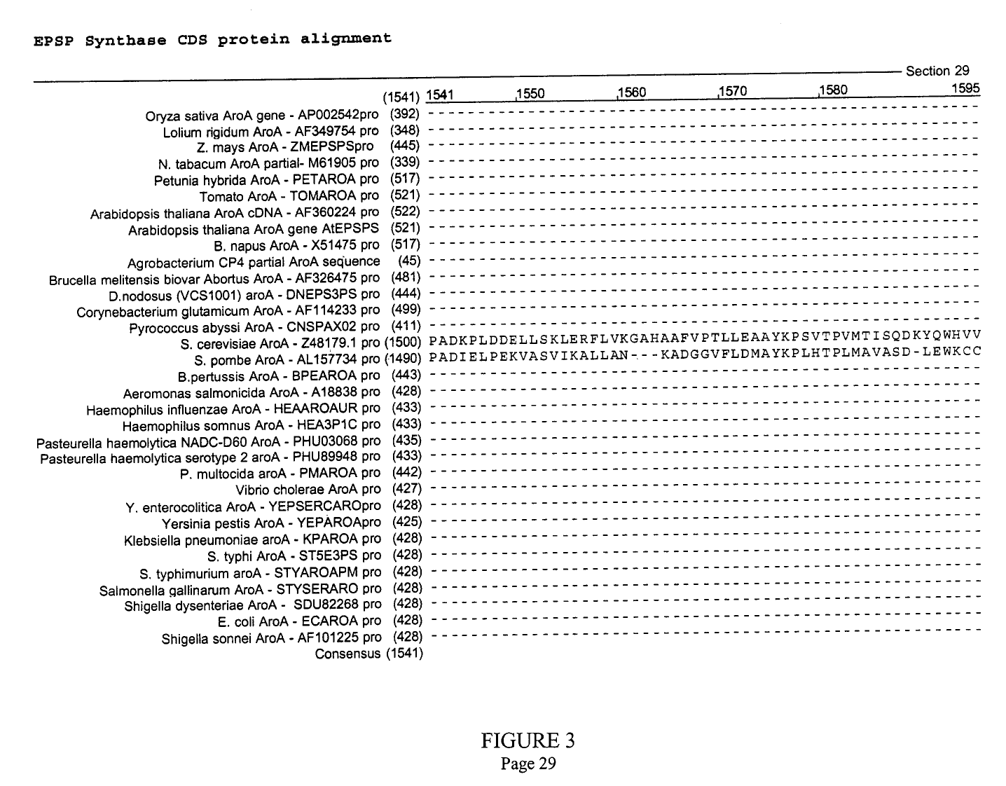

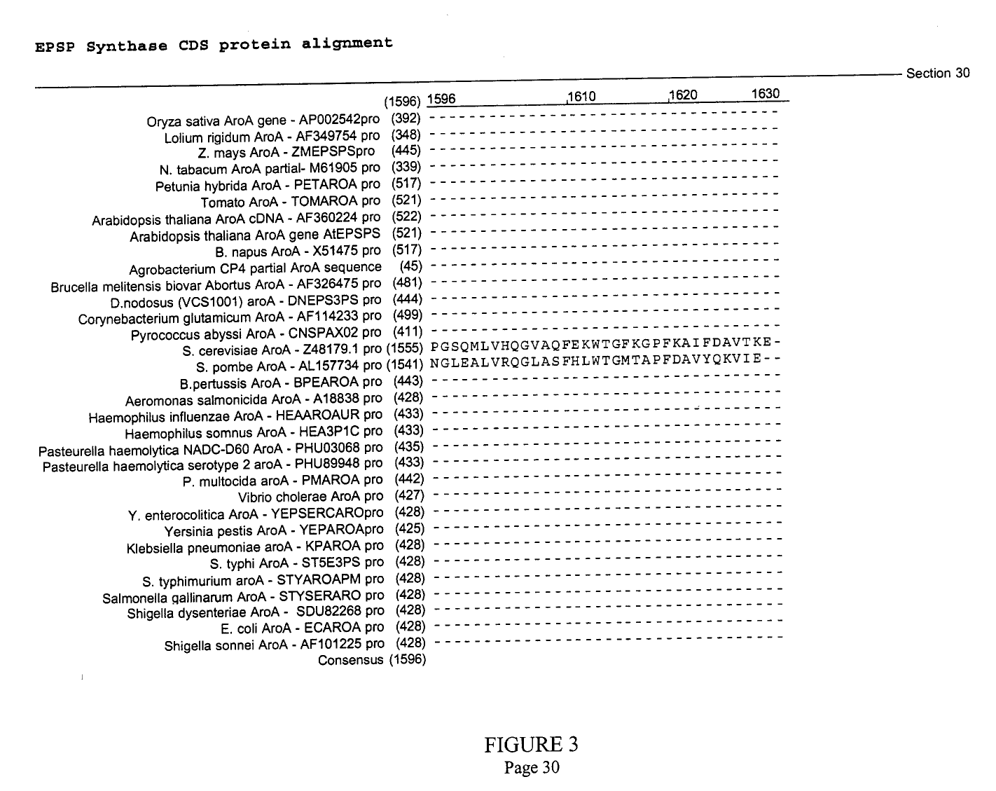

The foregoing mutations in the EPSPS gene are seen in the Arabidopsis thaliana EPSPS gene and protein sequences in FIG. 1. The present invention also encompasses mutant EPSPS genes of other plant species (homologs). However, due to variations in the EPSPS genes of different species, the position number of the amino acid residue to be changed in one species may be different in another species. Nevertheless, the analogous position is readily identified by one of skill in the art by sequence homology. For example, FIG. 3 shows the aligned amino acid sequences of homologs of the EPSPS gene in various organisms including, Arabidopsis thaliana, Zea mays, Petunia hybrida, N. tabacum, tomato and Brassica napus. Thus, the analogous positions in Zea mays are Asp₅₁, Gly₁₀₁, Arg₁₃₁, Arg₃₆₂, His₄₀₃, Arg₄₀₄ and Lys₄₂₉. Thus, the Zea mays EPSPS amino acid sequence is mutated at one or more of the following amino acid positions and results in one or more of the following substitutions:

(i) Asp₅₁—Glu

(ii) Gly₁₀₁—Ser or Met

(iii) Arg131—Glu

(iv) Arg362—Lys

(v) His₄₀₃—Leu or Arg

(vi) His₄₀₃Arg₄₀₄—Arg₄₀₃Lys₄₀₄

(vii) Lys₄₂₉—Arg

In Brassica napus, the analogous amino acid positions are D₁₂₂, R₂₀₃, R434, H₄₇₅, R476, G₁₇₃ and K₅₀₁. Thus, the Brassica napus EPSPS amino acid sequence is mutated at one or more of the following amino acid positions and results in one or more of the following substitutions:

(i) Asp₁₂₂—Glu

(ii) Arg203—Glu

(iii) Arg434—Lys

(iv) His₄₇₅—Leu or Arg

(v) His₄₇₅Arg₄₇₆—Arg₄₇₅Lys₄₇₆

(vi) Gly₁₇₃—Met or Ser

(vii) Lys₅₀₁—Arg

In Petunia hybrida the analogous positions are D₁₂₂, R₂₀₃, R434, H₄₇₅, R476, G₁₇₃ and K₅₀₁. Thus, the Petunia hybrida EPSPS amino acid sequence is mutated at one or more of the following amino acid positions and results in one or more of the following substitutions:

(i) Asp₂₂—Glu

(ii) Arg203—Glu

(iii) Arg₄₃₄—Lys

(iv) His₄₇₅—Leu or Arg

(v) His₄₇₅Arg₄₇₆—Arg₄₇₅Lys₄₇₆

(vi) Gly₁₇₃—Met or Ser

(vii) Lys₅₀₁—Arg

5.3 The Delivery of Recombinagenic Oligonucleobases into Plant Cells

Any commonly known method can be used in the methods of the present invention to transform a plant cell with a recombinagenic oligonucleobases. Illustrative methods are listed below.

5.3.1 Microcarriers and Microfibers

The use of metallic microcarriers (microspheres) for introducing large fragments of DNA into plant cells having cellulose cell walls by projectile penetration is well known to those skilled in the relevant art (henceforth biolistic delivery). U.S. Pat. Nos. 4,945,050; 5,100,792 and 5,204,253 describe general techniques for selecting microcarriers and devices for projecting them. U.S. Pat. Nos. 5,484,956 and 5,489,520 describe the preparation of fertile transgenic corn using microprojectile bombardment of corn callus tissue. The biolistic techniques are also used in transforming immature corn embryos.

Specific conditions for using microcarriers in the methods of the present invention are described in International Publication WO 99/07865. In an illustrative technique, ice cold microcarriers (60 mg/ml), mixed duplex oligonucleotide (60 mg/ml) 2.5 M CaCl₂ and 0.1 M spermidine are added in that order; the mixture is gently agitated, e.g., by vortexing, for 10 minutes and let stand at room temperature for 10 minutes, whereupon the microcarriers are diluted in 5 volumes of ethanol, centrifuged and resuspended in 100% ethanol. Good results can be obtained with a concentration in the adhering solution of 8-10 μg/μl microcarriers, 14-17 μg/ml mixed duplex oligonucleotide, 1.1-1.4 M CaCl₂ and 18-22 mM spermidine. Optimal results were observed under the conditions of 8 μg/μl microcarriers, 16.5 μg/ml mixed duplex oligonucleotide, 1.3 M CaCl₂ and 21 mM spermidine.

Recombinagenic oligonucleobases can also be introduced into plant cells for the practice of the present invention using microfibers to penetrate the cell wall and cell membrane. U.S. Pat. No. 5,302,523 to Coffee et al. describes the use of 30×0.5 μm and 10×0.3 μm silicon carbide fibers to facilitate transformation of suspension maize cultures of Black Mexican Sweet. Any mechanical technique that can be used to introduce DNA for transformation of a plant cell using microfibers can be used to deliver recombinagenic oligonucleobases for use in making the present EPSPS mutants. The process disclosed by Coffee et al in U.S. Pat. No. 5,302,523 can be employed with regenerable plant cell materials to introduce the present recombinagenic oligonucleobases to effect the mutation of the EPSPS gene whereby a whole mutated plant can be recovered that exhibits the glyphosate resistant phenotype.

An illustrative technique for microfiber delivery of a recombinagenic oligonucleobase is as follows: Sterile microfibers (2 μg) are suspended in 150 μl of plant culture medium containing about 10 μg of a mixed duplex oligonucleotide. A suspension culture is allowed to settle and equal volumes of packed cells and the sterile fiber/nucleotide suspension are vortexed for 10 minutes and plated. Selective media are applied immediately or with a delay of up to about 120 hours as is appropriate for the particular trait.

5.3.2 Electroporation

In an alternative embodiment, the recombinagenic oligonucleobases can be delivered to the plant cell by electroporation of a protoplast derived from a plant part. The protoplasts are formed by enzymatic treatment of a plant part, such as a leaf, according to techniques well known to those skilled in the art. See, e.g., Gallois et al., 1996, in Methods in Molecular Biology 55:89-107, Humana Press, Totowa, N.J.; Kipp et al., 1999, in Methods in Molecular Biology 133:213-221, Humana Press, Totowa, N.J. The protoplasts need not be cultured in growth media prior to electroporation. Illustrative conditions for electroporation are 3×10⁵ protoplasts in a total volume of 0.3 ml with a concentration of recombinagenic oligonucleobase of between 0.6-4 μg/mL.

Recombinagenic oligonucleobases can also be introduced into microspores by electroporation. Upon release of the tetrad, the microspore is uninucleate and thin-walled. It begins to enlarge and develops a germpore before the exine forms. A microspore at this stage is potentially more amenable to transformation with exogenous DNA than other plant cells. In addition, microspore development can be altered in vitro to produce either haploid embryos or embryogenic callus that can be regenerated into plants (Coumans et al., Plant Cell Rep. 7:618-621, 1989; Datta et al., Plant Sci. 67:83-88, 1990; Maheshwari et al., Am. J Bot. 69:865-879, 1982; Schaeffer, Adv. In Cell Culture 7:161-182, 1989; Swanson et al., Plant Cell Rep. 6:94-97, 1987). Thus, transformed microspores can be regenerated directly into haploid plants or dihaploid fertile plants upon chromosome doubling by standard methods. See also co-pending application U.S. Ser. No. 09/680,858 entitled Compositions and Methods for Plant Genetic Modification which is incorporated herein by reference.

Microspore electroporation can be practiced with any plant species for which microspore culture is possible, including but not limited to plants in the families Graminae, Leguminoceae, Cruciferaceae, Solanaceac, Cucurbitaceae, Rosaccae, Poaceae, Lilaceae, Rutaceae, Vitaceae, including such species as corn (Zea mays), wheat (Triticum aestivum), rice (Oryza sativa), oats, barley, canola (Brassica napus, Brassica rapa, Brassica oleracea, and Brassicajuncea), cotton (Gossypium hirsuitum L.), various legume species (e.g., soybean [Glycine max], pea [Pisum sativum], etc.), grapes [Vitis vinifera], and a host of other important crop plants. Microspore embryogenesis, both from anther and microspore culture, has been described in more than 170 species, belonging to 68 genera and 28 families of dicotyledons and monocotyledons (Raghavan, Embryogenesis in Agniosperms: A Developmental and Experimental Study, Cambridge University Press, Cambridge, England, 1986; Rhagavan, Cell Differentiation 21:213-226, 1987; Raemakers et al., Euphytica 81:93-107, 1995). For a detailed discussion of microspore isolation, culture, and regeneration of double haploid plants from microspore-derived embryos [MDE] in Brassica napus L., see Nehlin, The Use of Rapeseed (Brassica napus L.) Microspores as a Tool for Biotechnological Applications, doctoral thesis, Swedish University of Agricultural Sciences, Uppsala, Sweden, 1999; also Nehlin et al., Plant Sci. 111:219-227, 1995, and Nehlin et al., Plant Sci. 111:219-227, 1995). Chromosome doubling from microspore or anther culture is a well-established technique for production of double-haploid homozogous plant lines in several crops (Heberle-Bors et al., In vitro pollen cultures: Progress and perspectives. In: Pollen Biotechnology. Gene expression and allergen characterization, vol. 85-109, ed. Mohapatra, S. S., and Knox, R. B., Chapman and Hall, New York, 1996).

Microspore electroporation methods are described in Jardinaud et al., Plant Sci. 93:177-184, 1993, and Fennell and Hauptman, Plant Cell Reports 11:567-570, 1992. Methods for electroporation of MDON into plant protoplasts can also be adapted for use in microspore electroporation.

5.3.3 Whiskers and Microinjection

In yet another alternative embodiment, the recombinagenic oligonucleobase can be delivered to the plant cell by whiskers or microinjection of the plant cell. The so called whiskers technique is performed essentially as described in Frame et al., 1994, Plant J. 6:941-948. The recombinagenic oligonucleobase is added to the whiskers and used to transform the plant cells. The recombinagenic oligonucleobase may be co-incubated with plasmids comprising sequences encoding proteins capable of forming recombinase complexes in plant cells such that recombination is catalyzed between the oligonucleotide and the target sequence in the EPSPS gene.

5.4 Selection of Glyphosate Resistant Plants

Plants or plant cells can be tested for resistance or tolerance to a phosphonomethylglycine herbicide using commonly known methods in the art, e.g., by growing the plant or plant cell in the presence of a herbicide and measuring the rate of growth as compared to the growth rate of control plants in the absence of the herbicide. In the case of glyphosate concentrations of from about 0.01 to about 20 mM are employed in selection medium.

### 6. EXAMPLE 1

### Production of Glyphosate-Resistant Arabidopsis EPSPS Genes

The following experiments demonstrate the production of mutant Arabidopsis thaliana EPSPS genes which are resistant to the herbicide glyphosate and which allows the plant cells to maintain a growth rate

6.1 Material and Methods

6.1.1 Isolation Of Arabidopsis Thaliana EPSPS cDNA

A 1.3 kb DNA fragment was amplified by PCR from an Arabidopsis cDNA library using the primers AtEXPEXPM1 and AtEXPEXP2CM-2. The two primers were designed to amplify the cDNA from the mature peptide to the termination codon. The 5′ primer AtEXPEXPM1 contains an XbaI site (underlined) and the 3′ primer AtEXPEXP2CM-2 contains a BglII site (underlined), sites which will be of use for cloning of the fragment into the expression vector.

AtEXPEXPM1

| AtEXPEXPM1 | |
|---|---|
| 5′-GCTCTAGAGAAAGCGTCGGAGATTGTACTT-3′ | (SEQ ID NO:40) |
| AtEXPEXP2CM-2 | |
| 5′-GCAGATCTGAGCTCTTAGTGCTTTGTGATTCTT | (SEQ ID NO:41) |
| TCAAGTAC-3′ | |

The PCR band was excised from the agarose gel and purified (GeneClean, Biol). Its sequence was then confirmed as the mature peptide sequence of Arabidopsis thaliana EPSPS gene.

6.1.2 Preparation of the Expression Vector

The EPSPS coding region of the AroE Bacillus subtilis gene was obtained by PCR using the following primers:

| BsAroE5′Xba | |
|---|---|
| 5′-GCGTCTAGAAAAACGAGATAAGGTGCAG-3′ | (SEQ ID NO:42) |
| and | |
| BsAroE3′BamHI | |
| 5′-GCGGATCCTCAGGATTTTTTCGAAAGCTTATTT | (SEQ ID NO:43) |
| AAATG-3′. | |

The PCR fragment, lacking an initiation codon (ATG), was cloned in-frame to the pACLacIMH6RecA vector by replacing the ORF of RecA by digesting with XbaI and BamHI. PACLacIMH6RecA contained the LacI region of Pet21 at positions 1440 to 3176, the MH6 RecA at positions 3809 to 5188, chloramphenicol resistance gene at positions 5445-218 (5446 to 5885 and 1 to 218), and the p15A origin of replication at positions 581 to 1424. The coding region of RecA gene was cloned from E. coli in-frame with the start codon and 6 histidine linker (MH6) behind the LacZ promoter of pUC19.

6.1.3 Cloning of the Arabidopsis EPSPS Gene

### Into Bacterial Expression Vector

The Arabidopsis 1.3 kb PCR fragment was digested with XbaI and BamHI (compatible with BglII) and cloned into the plasmid pACYCLacIMH6EPSPS, in place of the Bacillus gene.

The clones obtained (selected on chloramphenicol) were then sequenced and confirmed positive. Confirmed clones are selected and the junctions between the cDNA and the cloning plasmid are confirmed to be identical to the expected sequences.

6.1.4 Novel Point Mutations in the EPSPS Gene

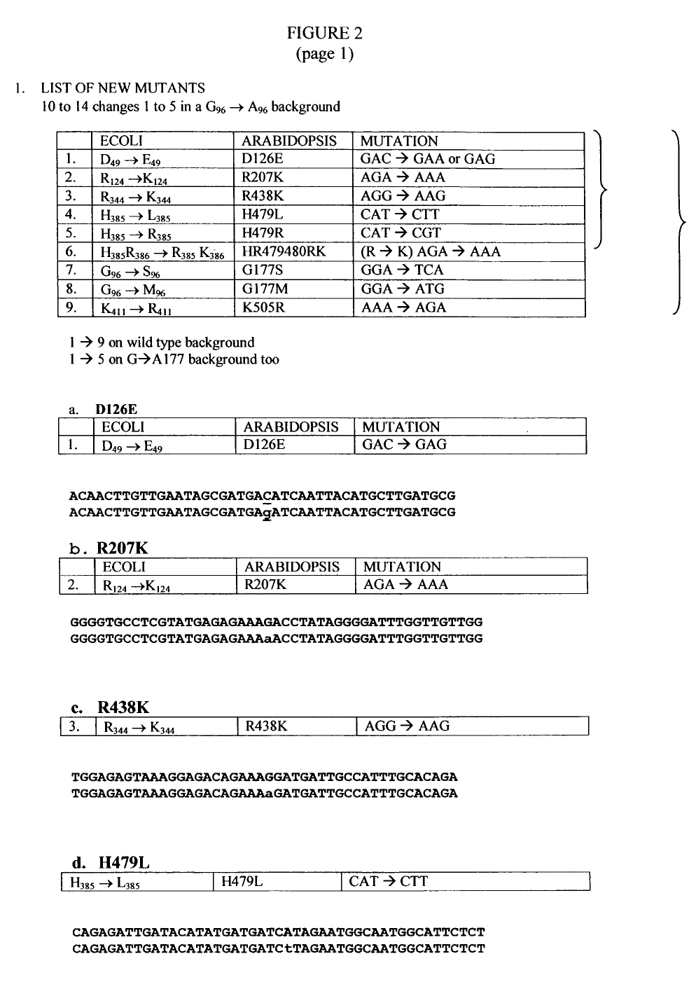

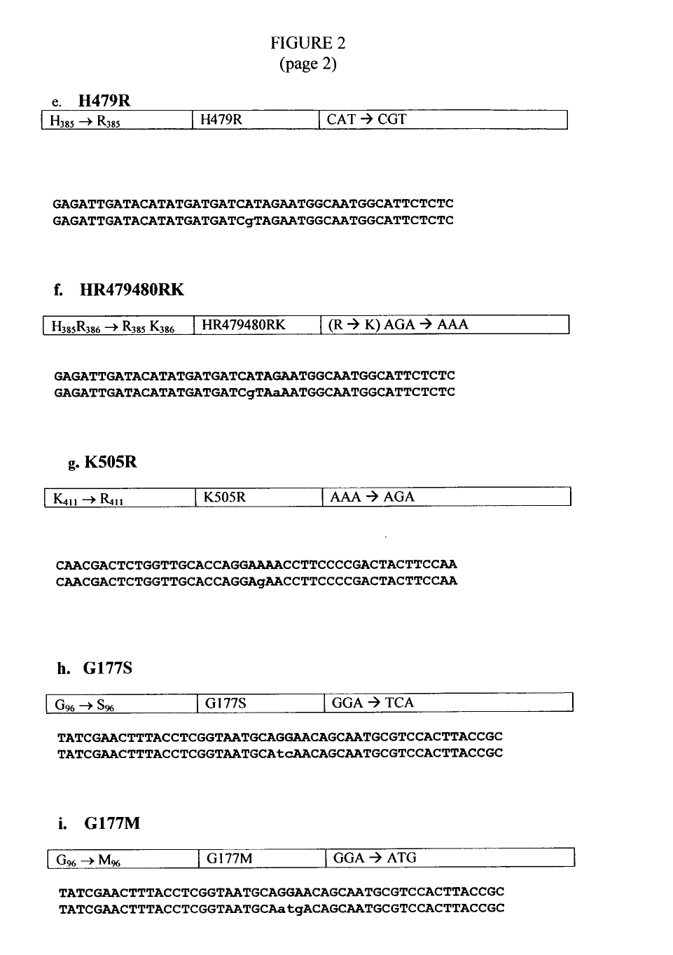

Ten different mutants of the Arabidopsis thaliana EPSPS gene were designed, (see FIG. 2). For the mutagenesis experiments, PCR primers were designed with one, two or three mutations. The PCR reactions are performed using a regular flanking primer (5′ ATEPS-198: 5′-GAAAGCGTCGGAGATTGTAC-3′) and one of the mutation-carrying primers that correspond to the mutations in FIG. 2.

The 353 bp PCR fragments obtained are purified (Qiagen PCR Purification kit) and their sequence confirmed. The fragments are then digested with PstI (underlined in the primer sequences) and BamHI and ligated to the pAtEPS-12 vector, which had itself been previously digested with PstI and BamHI.JM109 (Promega) competent cells are used for the transformation and plated onto chloramphenicol-containing LB plates. Clones from each mutagenesis experiment are then isolated and their sequence confirmed.

6.1.5 Glyphosate Resistance Assays

Electrocompetent cells of SA4247, a LacZ—Salmonella typhi strain, are prepared according to well known procedures (see Current Protocols in Molecular Biology, (Wiley and Sons, Inc.)). 30 μl of SA4247 competent cells are electroporated with 20 ng of each plasmid DNA encoding Arabidopsis wild-type and mutant EPSPS proteins, Bacillus wild-type EPSPS, along with a mock transfection as a control. The settings for electroporation are 25° F., 2.5 KV and 200 ohms. After electroporation, the cells are transferred into a 15 ml culture tube and supplemented with 970 μl of SOC medium. The cultures are incubated for 1½ hours at 37° C. at 225 rpm. 50 μl of each culture are plated onto LB plates containing 17 μg/ml chloramphenicol (in duplicates) and incubated overnight at 37° C. On the following day, 5 colonies of each plate are picked and transferred onto M9 plates and incubated overnight at 37° C.

Colonies from the overnight incubation on solid M9 are inoculated into 4 ml of liquid M9 medium and grown overnight at 37° C. On the following day, 25 ml of liquid M9 medium containing chloramphenicol, IPTG and 17 mM or 0 mM Glyphosate (Aldrich, 33775-7) are inoculated with 1-2 ml of each overnight culture (in duplicates), the starting OD (at 600 nm) is measured and all the cultures are normalized to start at the same OD. An OD measurement is taken every hour for seven hours. As a control of the bacterial growth, a culture of untransformed Salmonella is also inoculated into plain LB medium.

6.1.7 Isolation and Purification of the Expressed Protein From Bacterial Clones

One milliliter of overnight culture of each of the bacterial clones is inoculated into 100 ml of liquid LB medium containing chloramphenicol. The cells are allowed to grow at 37° C. until they reach an OD of 0.5-0.7 (approximately 3½ hours). IPTG is then added to the cultures to a concentration of 1.0 mM. The cells are grown five additional hours. They are then pelleted at 4000 rpm for 20 minutes at 4° C.

The isolation and the purification of the His-tagged proteins are performed following the Qiagen Ni-NTA Protein Purification System. Cell lysates and eluates are run in duplicates on 12.5% acrylamide gels. One of the gels is silver-stained for immediate visualization, the second gel is transferred onto Millipore Immobilon-P membrane, and blocked overnight in 5% milk in TBS-T. The membrane is then exposed to Anti-His primary antibody solution (Amersham Pharmacia biotech, cat#37-4710), followed by exposure to Anti-Mouse-IgG secondary antibody solution. (NIF825, from Amersham Pharmacia biotech ECLWestern blotting anlysis system, cat# RPN2108). Washes and detection reactions are performed according to the manufacturer instructions. Autoradiograms are developed after 5 minutes exposure.

### 7. EXAMPLE

### Microprojectile Bombardment of a Tobacco (NT-1) Cell Suspension

For microprojectile bombardment of plant cells, the media and protocols found in Gelvin, S. B., et al., (eds) 1991, Plant Molecular Biology Manual (Kluwer Acad. Pub.) are followed. Gold particles are coated with a recombinagenic oligonucleobase according the following protocol. The microprojectiles are first prepared for coating, then immediately coated with the recombinagenic oligonucleobase. To prepare the microprojectiles, suspend 60 mg of gold particles in 1 ml of 100% ethanol. Sonicate the suspension for three, 30 sec bursts to disperse the particles. Centrifuge at 12,000×g for 30 sec, then discard the supernatant. Add 1 ml of 100% ethanol, vortex for 15 sec, centrifuge at 12,000×g for 5 min, then discard the supernatant. A 25 μl suspension of washed gold particles (1.0 μm diameter, 60 mg/ml) in H₂O is slowly vortexed, then 40 μl MDON (50 μg/ml), 75 μl of 2.5 M CaCl₂, 75 μl 0.1M spermidine are sequentially added to the suspension. All solutions are ice cold. The completed mixture is vortexed for a further 10 min and the particles are allowed to settle at room temperature for a further 10 min. The pellet is washed in 100% ethanol and resuspended in 50 μl of absolute ethanol. Biolistic delivery is performed using a Biorad Biolistic gun with the following settings: tank pressure 1100 psi, rupture disks×2 breaking at 900 psi, particle suspension volume 5 μl.

Lawns of NT-1 cells of approximately 5 cm in diameter, containing approximately 5 million cells, are grown for three days on standard media at 28° C. Gold particles are coated with a recombinagenic oligonucleobase and shot as above. The cells are cultured a further 2.5 days, suspended and transferred to solid medium supplemented with from about 0.01-20 mM glyphosate for selection of glyphosate-resistant mutant cells.

For more stringent selection of glyphosate-resistant cells, cells are transferred from each bombarded plate to 15 ml tubes containing 5 ml of liquid NT-1 cell suspension medium (CSM: Murashige and Skoog salts [Gibco BRL, Grand Island, N.Y.], 500 mg/l MES, 1 mg/l thiamine, 100 mg/l myoinositol, 180 mg/l KH₂PO₄, 2.21 mg/L 2,4-diclorophenoxyacetic acid [2,4-D], 30 g/L sucrose, pH 5.7) 2 d after bombardment. The tubes are inverted several times to disperse cell clumps. The cells are then transferred to solidified CSM medium (CSM with add 8 g/l agar-agar [Sigma, St. Louis, Mo.]) containing 0.01-20 mM glyphosate. After an appropriate period for selection, actively growing cells (raised, light-colored colonies) are selected and transferred to solidified CSM media containing 0.01-20 mM glyphosate. Three to four weeks later, actively growing cells are selected, then transferred to solidified CSM containing 0.01-20 mM glyphosate. Cells that survive this treatment are then analyzed to determine if they have the mutated EPSPS gene.

### 8. EXAMPLE

### Electroporation of Tobacco Mesophyll Protoplasts

Leaves are harvested from 5- to 6-week-old in vitro-grown tobacco plantlets. For protoplast isolation, the procedure of Gallois et al. (1996, Electroporation of tobacco leaf protoplasts using plasmid DNA or total genomic DNA. Methods in Molecular Biology, Vol. 55: Plant Cell Electroporation and Electrofusion Protocols Edited by: J. A. Nickoloff Humana Press Inc., Totowa, N.J. pp. 89-107) is used. The following enzyme solution is used: 1.2% cellulase R-10 “Onozuka” (Karlan, Santa Rosa, Calif.), 0.8% macerozyme R-10 (Karlan, Santa Rosa, Calif.), 90 g/l mannitol, 10 mM MES, filter sterilize, store in 10 ml aliquots at −20° C. Leaves are cut from the mid-vein out every 1-2 mm. They are then placed abaxial side down in contact with 10 ml of enzyme solution in a 100×20 mm petri plate. A total of 1 g of leaf tissue is placed in each plate, and the plates are incubated at 25° C. in the dark for 16 hr. The digested leaf material is pipetted and sieved through a 100 μm nylon screen cloth (Small Parts, Inc., Miami Lakes, Fla.). The filtrate is then transferred to a centrifuge tube and centrifuged at 1,000 rpm for 10 min. All centrifugations for this protocol are performed similarly. The protoplasts collect in a band at the top. The band of protoplasts is then transferred to a clean centrifuge tube to which 10 ml of a washing solution (0.4 M sucrose and 80 mM KCl) is added. The protoplasts are gently resuspended, centrifuged, then washed again. After the last wash, the protoplast density is determined by dispensing a small aliquot onto a hemocytometer.

For electroporation, the protoplasts are resuspended to a density of 1×10⁶ protoplasts/ml in electroporation buffer (80 mM KCl, 4 mM CaCl₂, 2 mM potassium phosphate, pH 7.2, 8% mannitol). The protoplasts are allowed to incubate at 8° C. for 2 hr. After 2 hr, 0.3 ml (3×10⁵ protoplasts) are transferred to each 0.4 cm cuvette, then placed on ice. GFP-2 (0.6-4 μg/mL) is added to each cuvette except for an unelectroporated control. The protoplasts are electroporated (250V, capacitance 250 μF., and time constant 10-14 ms). The protoplasts are allowed to recover for 10 min on ice, then transferred to petri plates (100×20 mm). After 35 min, 10 ml of POM (80% [v/v] CSM, 0.3M mannitol, 20% [v/v] supernatant from the initial centrifugation of the NT-1 cell suspension prior to protoplast isolation), is added to each plate. The plates are transferred to the dark at 25° C. for 24 hr, then transferred to the light. The protoplast cultures are then maintained according to Gallois supra.

### 9. EXAMPLE

### Canola Microspore Isolation, Electroporation, and Embryogenesis

For microspore isolation, canola (Brassica napus or Brassica rapa) buds of appropriate size (depending on environmental conditions: 12-20° C., 3.5-4.5 mm; 20-23° C., 3.0-3.5 mm; 23-28° C., 2.2-2.8 mm) are picked from approximately 6-10 racemes for a small culture or up to 50 for a large culture. The buds are then placed in a steel sterilization basket. In the hood, buds are sterilized by submersing the sterilization baskets containing the buds into 200 ml of 5.6% bleach for 10 minutes. The sterile buds are then rinsed with 200 ml of cold, sterile water for 5 minutes, twice. The buds are then transferred from the sterilization baskets to a blender cup and 25-30 ml of cold microspore wash (13% sucrose solution, pH 6.0) is added. The buds are homogenized with a blender by alternating high and low speeds, five seconds each, for a total of 20 seconds. (Alternatively, the buds are transferred to the mortar, 30 ml of microspore wash are added, and the tissues are ground up using a pestle for approximately 20 sec.) The contents of the blender cup are poured through nested 63 um and 44 um sterile filters in a beaker-funnel apparatus. The blender cup is then rinsed with 10-15 ml microspore wash. The filtrate is poured into 50 ml plastic centrifuge tubes and the volume is adjusted to 50 ml with microspore wash. The tubes are centrifuged for five minutes at 200×g. After centrifugation, the dark green supernatant is decanted, leaving a yellow spore pellet at the bottom. The wash procedure is repeated two more times for a total of three centrifugations. The supernatant should become clearer with each wash step. The first two cycles of washing should be done in less than 10 minutes to avoid autotoxicity. After the third spin, the microspores are resuspended in 50 ml of NLN liquid culture medium (less NLN can be used, depending on pellet size, to permit an easier volume adjustment after determining initial microspore concentration). To make NLN Medium, combine 0.125 g KNO₃, 1.25 g MgSO₄ 7H₂O, 0.5 g Ca(NO₃)₂ 4H₂O, 0.125 g KH₂PO₄, and 4 ml FeSO₄ EDTA [per 500 ml: 1.39 g FeSO₄ 7H₂O, 1.865 g Na₂ EDTA]. Add 10 ml 100× NN vitamin stock [per L: 0.005 g biotin, 0.05 g folic acid, 0.2 g glycine, 10.0 g myoinositol, 0.5 g nicotinic acid, 0.05 g pyridoxine HCl, 0.05 g thiamine HCI], 10 ml 100× MS micronutrient stock [per L: 2.23 g MnSO₄ 4H₂O, 0.62 g boric acid, 0.86 g ZnSO₄·7H₂O, 0.025 g Na₂MoO₄ 2H₂O, 0.0025 g CuSO₄ 5H₂O, 0.0025 g CoCl₂·6H₂O], 0.03 g glutathione [reduced form], 0.8 g L-glutamine, 0.1 g L-serine, 130 g sucrose, and adjust the pH to 6.0.

Microspores are electroporated using the protoplast electroporation procedure detailed above for Brassica napus or Brassica rapa. For Brassica or other species, other well-known microspore electroporation protocols can be used, including those provided by manufacturers for use with electroporation equipment, e.g., the Electro Cell Manipulator® (ECM 600, BTX Division of Genetronics) or Electro Square Porator™ (T820, BTX Division of Genetronics).

For example, for Zea mays, the following protocol is provided for use with the Electro Square Porator™ (T820, BTX Division of Genetronics). Pollen is collected from greenhouse-grown plants. Supplemental light is provided by high-pressure 400 W sodium lights with an average output of 500 ft-candles to achieve a 16 hr/daylight period. Tassles are shaken the day before electroporation to remove old pollen and to ensure collection of recently mature pollen the next morning. Pollen is germinated for 3-5 minutes before electroporation in 0.20 M sucrose, 1.27 mM Ca(NO₃)₂ 4H₂O, 0.16 mM H₃BO₃, 0.99 mM KNO₃, pH 5.2. The following electorporation settings are used: HV Mode/3 KV, one pulse of 99 μsec pulse length at a voltage of 1.5 kV and field strength of 3.75 kV/cm using a disposable cuvette (p/n 640) with a 4 mm gap. Electroporation is carried out at room temperature using a sample volume of 800 μl.

The following protocol is employed to achieve embryogenesis of the microspores. A hemacytometer is used to determine the microspore concentration at the initial volume by counting all microspores in each of the corner quadrants of the hemacytometer. The new culture is determined using the following equation: (number of cells counted/number of fields counted) (10,000) (initial volume/100,000)=new volume. The required culture density for microspores is between 80,000 and 100,000 spores per ml. The volume of the culture is adjusted accordingly and the culture is mixed well. 15 ml of the culture is pipetted into an appropriate number of petri plates. For even plating, one can make slight adjustments (usually no more than 2-3 ml) to make the culture volume a factor of 15, resulting in even plating. Plates are sealed with a double layer of parafilm and stacked in a 30° C. incubator in the dark. After seven days, the plates are observed under an inverted scope to look for cell divisions and embryo development. If cell divisions and tiny globular embryos are observed, the plates are returned to the incubator for another seven days. Otherwise, the culture is discarded. After 14 days at 30 C, the plates are placed on a shaker at 50 rpm at room temperature in the dark for an additional 14 days. After 28-35 days of culture, embryos should be approximately 5 mm long with visible cotyledons. Embryos are then transferred to solid B5 germination medium and exposed to a temperature of 4° C. immediately after transfer to solid medium to increase the yield of mature embryos. To make B5 solid germination medium,_combine 400 ml B5×10 Stock (per 4 L: 50 g KNO_{3, 5} g MgSO₄ 7H₂O, 15 g CaCl₂ 2H₂O, 2.68 g (NH₄)2SO₄, 3 g NaH₂PO₄H₂O, 32 ml FeSO₄ EDTA), 200 ml B5 vitamin stock [per L: 10 g myoinositol, 0.1 g nicotinic acid, 0.1 g pyridoxine HCl, 1 g thiamine-HCl], 200 ml 100×B5 micronutrient stock [per L: 1 g MnSO₄H₂O, 0.3 g H₃BO₃, 0.2 g ZnSO₄ 7H₂O, 0.025 g Na₂MoO₄ 2H₂O, 0.0025 g CuSO₄ 5H₂O, 0.0025 g CoCl₂ 6H₂O], 20 ml KI stock [0.83 g/L KI]; 40 g sucrose; and 2 ml GA₃ stock [0.1 g/L GA]. Bring the volume up to 2 L with double distilled water, pH 5.7, and add 8 g agar per L before autoclaving. The embryos are maintained at 4° C. for 10 days. The plates are then moved to a light chamber set between 23 and 27° C. with a 12 hr light regime. The plates remain in these conditions for 30 days. The plantlets generated after this period can be transferred directly to soil.

The invention claimed and described herein is not to be limited in scope by the specific embodiments herein disclosed since these embodiments are intended as illustrations of several aspects of the invention. Indeed, various modifications of the invention in addition to those shown and described herein will become apparent to those skilled in the art from the foregoing description. Such modifications are also intended to fall within the scope of the appended claims.

A number of references are cited herein, the entire disclosures of which are incorporated herein, in their entirety, by reference.

We claim:

1. An herbicide resistant plant that expresses a mutant EPSPS gene product wherein the EPSPS gene is mutated at a position to change one or more amino acid positions in the gene product, said amino acid positions selected from the group consisting of Asp₁₂₆, Arg207, Arg438, His₄₇₉, Arg480, Gly₁₇₇ and Lys₅₀₅ in Arabidopsis or at an analogous amino acid position in an EPSPS homolog.

2. The plant according to claim 1 wherein the plant is Zea mays and the amino acid positions are selected from the group consisting of Asp₅₁, Gly₁₀₁, Arg₁₃₁, Arg₃₆₂, His₄₀₃, Arg₄₀₄ and Lys₄₂₉.

3. The plant according to claim 1 wherein the plant is Brassica napus and the amino acid positions are selected from the group consisting of Asp₁₂₂, Arg203, Arg434, His₄₇₅, Arg476, Gly₁₇₃ and Lys₅₀₁.

4. The plant according to claim 1 wherein the plant is Petunia hybrida and the amino acid positions are selected from the group consisting of Asp₁₂₂, Arg203, Arg434, His₄₇₅, Arg476, Gly₁₇₃ and Lys₅₀₁.

5. The plant according to claim 1 wherein the plant is selected from the group consisting of corn, wheat, rice, barley, soybean, cotton, sugarbeet, oilseed rape, canola, flax, sunflower, potato, tobacco, tomato, alfalfa, poplar, pine, eucalyptus, apple, lettuce, peas, lentils, grape and turf grasses.

6. The plant according to claim 1 in which the mutated gene results in one or more of the following amino acid substitutions in the EPSPS gene product in comparison with the wild-type sequence:
(i) Asp₁₂₆—Glu
(ii) Arg₂₀₇—Glu
(iii) Arg438—Lys
(iv) His₄₇₉—Arg or Leu
(v) His₄₇₉R₄₈₀—Arg₄₇₉Lys₄₈₀
(vi) Gly₁₇₇—Met or Ser
(vii) Lys₅₀₅—Arg

7. The plant according to claim 2 in which the mutated gene results in one or more of the following amino acid substitutions in the EPSPS gene product in comparison with the wild-type sequence:
(i) Asp₅₁—Glu
(ii) Gly₁₀₁—Ser or Met
(iii) Arg₁₃₁—Glu
(iv) Arg₃₆₂—Lys
(v) His₄₀₃—Leu or Arg
(vi) His₄₀₃Arg₄₀₄—Arg₄₀₃Lys₄₀₄
(vii) Lys₄₂₉—Arg

8. The plant according to claim 3 in which the mutated gene results in one or more of the following amino acid substitutions in the EPSPS gene product in comparison with the wild-type sequence:
(i) Asp₁₂₂—Glu
(ii) Arg₂₀₃—Glu
(iii) Arg₄₃₄—Lys
(iv) His₄₇₅—Leu or Arg
(v) His₄₇₅Arg₄₇₆—Arg₄₇₅Lys₄₇₆
(vi) Gly₁₇₃—Met or Ser
(vii) Lys₅₀₁—Arg.

9. The plant according to claim 4 in which the mutated gene results in one or more of the following amino acid substitutions in the EPSPS gene product in comparison with the wild-type sequence:
(i) Asp₁₂₂—Glu
(ii) Arg₂₀₃—Glu
(iii) Arg₄₃₄—Lys
(iv) His₄₇₅—Leu or Arg
(v) His₄₇₅Arg₄₇₆—Arg₄₇₅Lys₄₇₆
(vi) Gly₁₇₃—Met or Ser
(vii) Lys₅₀₁—Arg.

10. A mutant EPSPS protein comprising the amino acid sequence of the Arabidopsis EPSPS gene product depicted in FIG. 1 in which one or more amino acids selected from the group consisting of Asp 126, Arg207, Arg438, His₄₇₉, Gly₁₇₇ and Lys₅₀₅ (or at an analogous amino acid position in an EPSPS homolog) is changed to a different amino acid, which mutant EPSPS protein has increased resistance or tolerance to a phosphonomethylglycine herbicide.

11. The mutant EPSPS protein of claim 10 further comprising a change at amino acid position Arg₄₈₀ to a different amino acid when amino acid His₄₇₉ is also changed to a different amino acid.

12. The mutant EPSPS protein of claim 11 wherein His₄₇₉ is changed to Arg₄₇₉ and Arg₄₈₀ is changed to Lys₄₈₀.

13. The mutant EPSPS protein of claim 10 wherein Asp₁₂₆ is changed to Glu₁₂₆.

14. The mutant EPSPS protein of claim 10 wherein the Arg₂₀₇ is changed to Glu₂₀₇.

15. The mutant EPSPS protein of claim 10 wherein the Arg₄₃₈ is changed to Lys₄₃₈.

16. The mutant EPSPS protein of claim 10 wherein the His₄₇₉ is changed to Leu₄₇₉ or Arg₄₇₉.

17. The mutant EPSPS protein of claim 10 wherein the Gly₁₇₇ is changed to Ser₁₇₇ or Met₁₇₇.

18. The mutant EPSPS protein of claim 10 wherein the Lys₅₀₅ is changed to Arg₅₀₅.

19. A method for producing an herbicide resistant or tolerant plant which comprises:
a. introducing into a plant cell a recombinagenic oligonucleobase to produce a mutant EPSPS gene wherein the EPSPS gene is mutated at a position to change one or more amino acid positions in the gene product, said amino acid positions selected from the group consisting of Asp₁₂₆, Arg₂₀₇, Arg₄₃₈, His₄₇₉, Arg₄₈₀, Gly₁₇₇ and Lys₅₀₅ in Arabidopsis or at an analogous amino acid position in an EPSPS homolog.; and
b. identifying a cell having a mutated EPSPS gene.

20. The method of claim 19 wherein the mutated EPSPS gene results in one or more of the following amino acid substitutions in the EPSPS gene product in comparison with the wild-type sequence:
(i) Asp₁₂₆—Glu
(ii) Arg₂₀₇—Glu
(iii) Arg₄₃₈—Lys
(iv) His₄₇₉—Arg or Leu
(v) His₄₇₉R₄₈₀—Arg₄₇₉Lys₄₈₀
(vi) Gly₁₇₇—Met or Ser
(vii) Lys₅₀₅—Arg.

21. The method of claim 19 wherein plant is a Zea mays plant and the amino acid positions in the Zea mays homolog are selected from the group consisting of Asp₅₁, Gly₁₀₁, Arg₁₃₁, Arg₃₆₂, His₄₀₃, Arg₄₀₄ and Lys₄₂₉.

22. The method of claim 21 wherein the mutated EPSPS gene results in one or more of the following amino acid substitutions in the EPSPS gene product in comparison with the wild-type sequence:
(i) Asp₅₁—Glu
(ii) Gly₁₀₁—Ser or Met
(iii) Arg₁₃₁—Glu
(iv) Arg₃₆₂—Lys
(v) His₄₀₃—Leu or Arg
(vi) His₄₀₃Arg₄₀₄—Arg₄₀₃Lys₄₀₄
(vii) Lys₄₂₉—Arg.

23. The method of claim 19 wherein the plant is a Brassica napus plant and the amino acid positions in the Brassica napus homolog are selected from the group consisting of Asp₁₂₂, Arg₂₀₃, Arg₄₃₄, His₄₇₅, Arg₄₇₆, Gly₁₇₃ and Lys₅₀₁.

24. The method of claim 23 wherein the mutated EPSPS gene results in one or more of the following amino acid substitutions in the EPSPS gene product in comparison with the wild-type sequence:
(i) Asp₁₂₂—Glu
(ii) Arg₂₀₃—Glu
(iii) Arg₄₃₄—Lys
(iv) His₄₇₅—Leu or Arg
(v) His₄₇₅Arg₄₇₆—Arg₄₇₅Lys₄₇₆
(vi) Gly₁₇₃—Met or Ser
(vii) Lys₅₀₁—Arg.

25. The method of claim 19 wherein the plant is a Petunia hybrida plant and the amino acid positions in the Petunia hybrida are selected from the group consisting of Asp₁₂₂, Arg₂₀₃, Arg₄₃₄, His₄₇₅, Arg₄₇₆, Gly₁₇₃ and Lys₅₀₁.

26. The method of claim 25 wherein the mutated EPSPS gene results in one or more of the following amino acid substitutions in the EPSPS gene product in comparison with the wild-type sequence:
(i) Asp₁₂₂—Glu
(ii) Arg₂₀₃—Glu
(iii) Arg₄₃₄—Lys
(iv) His₄₇₅—Leu or Arg
(v) His₄₇₅Arg₄₇₆—Arg₄₇₅Lys₄₇₆
(vi) Gly₁₇₃—Met or Ser
(vii) Lys₅₀₁—Arg.

27. The method of claim 19 wherein the recombinagenic oligonucleobase is a mixed duplex nucleotide or a SSMOV.

28. The method of claim 27 wherein the mixed duplex nucleotide contains a first homologous region which has a sequence identical to the sequence of at least 6 base pairs of the first fragment of the target EPSPS gene and a second homologous region which has a sequence identical to the sequence of at least 6 based pairs of a second fragment of the target EPSPS gene, and an intervening region which contains at least one nucleobase heterologous to the target EPSPS gene, which intervening region connects the first and second homologous region.

29. The method of claim 19 wherein the recombinagenic oligonucleobase is introduced by electroporation.

30. The method of claim 19 in which the plant is selected from the group consisting of the plant may be selected from a species of plant from the group consisting of canola, sunflower, tobacco, sugar beet, cotton, maize, wheat, barley, rice, sorghum, tomato, mango, peach, apple, pear, strawberry, banana, melon, potato, sweet potato, yam, carrot, lettuce, onion, soya spp, sugar cane, pea, peanut, field beans, poplar, grape, citrus, alfalfa, rye, oats, turf grasses, forage grasses, flax, oilseed rape, cucumber, morning glory, balsam, pepper, eggplant, marigold, lotus, cabbage, daisy, carnation, tulip, iris, lily, nut producing plants, pine, eucalyptus, lentils, and other Brassica sp.

31. A method of making a glyphosate resistant plant which comprises:
a. providing a recombinagenic oligonucleobase to produce a mutant EPSPS gene wherein the EPSPS gene is mutated at a position to change one or more amino acid positions in the gene product, said amino acid positions selected from the group consisting of Asp1₁₂₆, Arg₂₀₇, Arg₄₃₈, His₄₇₉, Arg₄₈₀, Gly₁₇₇ and Lys₅₀₅ in Arabidopsis or at an analogous amino acid position in an EPSPS homolog;
b. introducing said recombinagenic oligonucleotide into a plant cell;
c. culturing said cell to obtain descendant plant cells, said descendant plant cells containing the mutant EPSPS gene; and
d. establishing that the mutant EPSPS gene is expressed in said descendant plant cells.

32. A method of making seeds that will grow into plants that are resistant to glyphosate herbicide which comprises:
a. providing a recombinagenic oligonucleobase to produce a mutant EPSPS gene wherein the EPSPS gene is mutated at a position to change one or more amino acid positions in the gene product, said amino acid positions selected from the group consisting of Asp₁₂₆, Arg₂₀₇, Arg₄₃₈, His₄₇₉, Arg₄₈₀, Gly₁₇₇ and Lys₅₀₅ in Arabidopsis or at an analogous amino acid position in an EPSPS homolog;
b. introducing said recombinagenic oligonucleotide into a plant cell;
c. culturing said cell to obtain descendant plant cells, said descendant plant cells containing the mutant EPSPS gene; and
d. establishing that the mutant EPSPS gene is expressed in said descendant plant cells
e. regenerating a whole fertile plant that expresses the mutant EPSPS gene; and
f. collecting the seed from the whole fertile plant.

33. The method of claim 32 wherein the seed is germinated to produce more seed containing the mutant EPSPS gene and glyphosate is applied to the germinated plants to kill any plants that do not contain the mutated EPSPS gene.

34. A method of selectively cultivating EPSPS mutant plants which comprises:
a. cultivating EPSPS mutant plants wherein the EPSPS gene is mutated at a position to change one or more amino acid positions in the gene product, said amino acid positions selected from the group consisting of Asp₁₂₆, Arg₂₀₇, Arg₄₃₈, His₄₇₉, Arg₄₈₀, Gly₁₇₇ and Lys₅₀₅ in Arabidopsis or at an analogous amino acid position in an EPSPS homolog;
b. applying a sufficient amount of glyphosate herbicide to the cultivated mutant plants of (a) such that the glyphosate is toxic to at least one non-mutant plant.

35. A method of propagating an EPSPS mutant plant wherein the EPSPS gene is mutated at a position to change one or more amino acid positions in the gene product, said amino acid positions selected from the group consisting of Asp₁₂₆, Arg₂₀₇, Arg₄₃₈, His₄₇₉, Arg₄₈₀, Gly₁₇₇ and Lys₅₀₅ in Arabidopsis or at an analogous amino acid position in an EPSPS homolog which comprises (1) vegetatively propagating a plant containing said EPSPS mutation or (2) culturing a plant cell or plant tissue containing said EPSPS mutation to form callus tissue and regenerating a plant therefrom wherein the regenerated plant contains said EPSPS mutation.
